(19) United States
(12) Patent Application Publication
(10) Pub. No.: US 2010/0048660 A1
(43) Pub. Date: Feb. 25, 2010

(54) TREATMENT OF DUCHENNE MUSCULAR DYSTROPHY

(76) Inventors: Graham Michael Wynne, Bicester (GB); Stephen Paul Wren, Rickmansworth (GB); Peter David Johnson, Oxford (GB); Paul Damien Price, Chesire (GB); Olivier De Moor, Oxford (GB); Gary Nugent, Didcot (GB); Richard Storer, Folkestone (GB); Richard Joseph Pye, Kidlington (GB); Colin Richard Dorgan, Abingdon (GB)

Correspondence Address: JONES DAY, 222 EAST 41ST ST, NEW YORK, NY, 10017, US

(21) Appl. No.: 12/278,771

(22) PCT Filed: Feb. 9, 2007
(86) PCT No.: PCT/GB2007/050056

§ 371 Date: Dec. 22, 2008

(30) Foreign Application Priority Data
Feb. 10, 2006 (GB) 0602767.6
Sep. 8, 2006 (GB) 0617737.2
Nov. 30, 2006 (GB) 0623984.2

(51) Int. Cl. A61K 31/4192; A61K 31/416; A61P 21/00

(52) U.S. Cl. 514/406; 514/359

(57) ABSTRACT

There are disclosed compound of Formula (I) or (II) wherein A¹, A², A³, A⁴ and A⁵, which may be the same or different, represent N or CR¹, R₉ represents -L-R³, in which L is a single bond or a linker group and R³ represents hydrogen or a substituent and in addition, when an adjacent pair of A¹-A⁴ each represent CR¹, then the adjacent carbon atoms, together with their substituents may form a ring B, when A⁵ represents CR¹, then A⁵ and N—R⁹, together with their substituents may form a ring C, or a pharmaceutically acceptable salt thereof, in the manufacture of a medicament for the therapeutic and/or prophylactic treatment of Duchenne muscular dystrophy, Becker muscular dystrophy or cachexia.

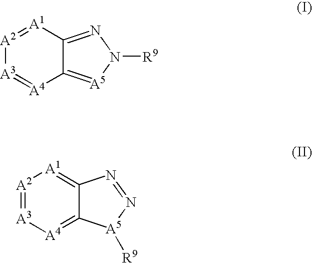

The present invention relates to a method of treatment of Duchenne muscular dystrophy.

Duchenne muscular dystrophy (DMD) is a common, genetic neuromuscular disease associated with the progressive deterioration of muscle function, first described over 150 years ago by the French neurologist, Duchenne de Boulogne, after whom the disease is named. DMD has been characterized as an X-linked recessive disorder that affects 1 in 3,500 males caused by mutations in the dystrophin gene. The gene is the largest in the human genome, encompassing 2.6 million base pairs of DNA and containing 79 exons. Approximately 60% of dystrophin mutations are large insertion or deletions that lead to frameshift errors downstream, whereas approximately 40% are point mutations or small frameshift rearrangements. The vast majority of DMD patients lack the dystrophin protein. Becker muscular dystrophy is a much milder form of DMD caused by reduction in the amount, or alteration in the size, of the dystrophin protein. The high incidence of DMD (1 in 10,000 sperm or eggs) means that genetic screening will never eliminate the disease, so an effective therapy is highly desirable.

A number of natural and engineered animal models of DMD exist, and provide a mainstay for preclinical studies (Allamand, V. & Campbell, K. P. Animal models for muscular dystrophy: valuable tools for the development of therapies. Hum. Mol. Genet. 9, 2459-2467 (2000).) Although the mouse, cat and dog models all have mutations in the DMD gene and exhibit a biochemical dystrophinopathy similar to that seen in humans, they show surprising and considerable variation in terms of their phenotype. Like humans, the canine (Golden retriever muscular dystrophy and German short-haired pointer) models have a severe phenotype; these dogs typically die of cardiac failure. Dogs offer the best phenocopy for human disease, and are considered a high benchmark for preclinical studies. Unfortunately, breeding these animals is expensive and difficult, and the clinical time course can be variable among litters.

The mdx mouse is the most widely used model due to availability, short gestation time, time to mature and relatively low cost (Bulfield, G., Siller, W. G., Wight, P. A. & Moore, K. J. X chromosome-linked muscular dystrophy (mdx) in the mouse. Proc. Natl. Acad. Sci. USA 81, 1189-1192 (1984)).

Since the discovery of the DMD gene about 20 years ago, varying degrees of success in the treatment of DMD have been achieved in preclinical animal studies, some of which are being followed up in humans. Present therapeutic strategies can be broadly divided into three groups: first, gene therapy approaches; second, cell therapy; and last, pharmacological therapy. Gene- and cell-based therapies offer the fundamental advantage of obviating the need to separately correct secondary defects/pathology (for example, contractures), especially if initiated early in the course of the disease. Unfortunately, these approaches face a number of technical hurdles. Immunological responses against viral vectors, myoblasts and newly synthesized dystrophin have been reported, in addition to toxicity, lack of stable expression and difficulty in delivery.

Pharmacological approaches for the treatment of muscular dystrophy differ from gene- and cell-based approaches in not being designed to deliver either the missing gene and/or protein. In general, the pharmacological strategies use drugs/molecules in an attempt to improve the phenotype by means such as decreasing inflammation, improving calcium homeostasis and increasing muscle progenitor proliferation or commitment. These strategies offer the advantage that they are easy to deliver systemically and can circumvent many of the immunological and/or toxicity issues that are related to vectors and cell-based therapies. Although investigations with corticosteroids and sodium cromoglycate, to reduce inflammation, dantrolene to maintain calcium homeostasis and clenbuterol to increase muscle strength, have produced promising results none of these potential therapies has yet been shown to be effective in treating DMD.

An alternative pharmacological approach is upregulation therapy. Upregulation therapy is based on increasing the expression of alternative genes to replace a defective gene and is particularly beneficial when an immune response is mounted against a previously absent protein. Upregulation of utrophin, an autosomal paralogue of dystrophin has been proposed as a potential therapy for DMD (Perkins & Davies, Neuromuscul Disord, S1: S78-S89 (2002), Khurana & Davies, Nat Rev Drug Discov 2:379-390 (2003)). When utrophin is overexpressed in transgenic mdx mice it localizes to the sarcolemma of muscle cells and restores the components of the dystrophin-associated protein complex (DAPC), which prevents the dystrophic development and in turn leads to functional improvement of skeletal muscle. Adenoviral delivery of utrophin in the dog has been shown to prevent pathology. Commencement of increased utrophin expression shortly after birth in the mouse model can be effective and no toxicity is observed when utrophin is ubiquitously expressed, which is promising for the translation of this therapy to humans. Upregulation of endogenous utrophin to sufficient levels to decrease pathology might be achieved by the delivery of small diffusible compounds.

We have now found a group of compounds which upregulate endogenous utrophin in predictive screens and, thus, may be useful in the treatment of DMD.

According to the invention, we provide use of a compound of Formula (I) or (II)

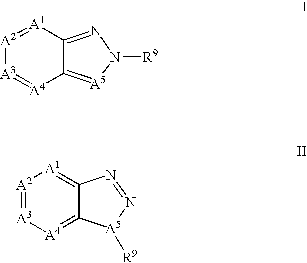

wherein
A¹, A², A³, A⁴ and A⁵, which may be the same or different, represent N or CR¹,
R⁹ represents -L-R³, in which L is a single bond or a linker group and R³ represents hydrogen or a substituent and
in addition,
when an adjacent pair of A¹-A⁴ each represent CR¹, then the adjacent carbon atoms, together with their substituents may form a ring B,
when A⁵ represents CR¹, then A⁵ and N—R⁹, together with their substituents may form a ring C,
or a pharmaceutically acceptable salt thereof,
in the manufacture of a medicament for the therapeutic and/or prophylactic treatment of Duchenne muscular dystrophy, Becker muscular dystrophy or cachexia.

When R⁹ represents H, compounds of formula I are tautomers of compounds of formula II.

Compounds of formula I may exist in tautomeric, enantiomeric and diastereomeric forms, all of which are included within the scope of the invention.

Certain compounds of formula I are novel. According to the invention, we also provide those compounds of formula I which are novel, together with processes for their preparation, compositions containing them, as well as their use as pharmaceuticals.

Some of the compounds falling within the scope of formula I are known, as such, but not as pharmaceuticals. According to the invention, we claim compounds known in the art as such, but not previously described for use as pharmaceuticals, as pharmaceuticals.

All of the compounds of formula I may be made by conventional methods. Methods of making heteroaromatic ring systems are well known in the art. In particular, methods of synthesis are discussed in Comprehensive Heterocyclic Chemistry, Vol. 1 (Eds.: A R Katritzky, C W Rees), Pergamon Press, Oxford, 1984 and Comprehensive Heterocyclic Chemistry II: A Review of the Literature 1982-1995 The Structure, Reactions, Synthesis, and Uses of Heterocyclic Compounds, Alan R. Katritzky (Editor), Charles W. Rees (Editor), E. F. V. Scriven (Editor), Pergamon Pr, June 1996. Other general resources which would aid synthesis of the compounds of interest include March's Advanced Organic Chemistry: Reactions, Mechanisms, and Structure, Wiley-Interscience; 5th edition (Jan. 15, 2001).

Compounds of formula I or pharmaceutically acceptable salts thereof may be prepared from a compound of formula II

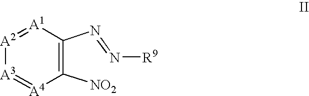

in which A¹, A², A³, and A⁴ are defined as above, in a reductive ring closure effected by reaction with thiourea-S,S-dioxide or a dithionite salt, for example an alkali metal salt, as described, for example, in EP 0 751 134. The reaction may be carried out in an aqueous solution, preferably an alcoholic aqueous solution, at a temperature of 60 to 80° C. Cyclisation will not occur in the presence of certain functionality, for example in the presence of —NH₂ or —OH functionality. These groups will need to be protected before cyclisation. For example —NH₂ groups may be protected as amides, and OH groups may be protected as ethers. Suitable protecting strategies are disclosed, for example, in EP 0 751 134.

Compounds of formula II may be prepared by a diazonium coupling reaction of a diazonium compound of formula III,

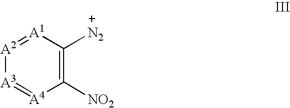

wherein A¹, A², A³, and A⁴ are defined as above, with phenyl derivatives of formula IV

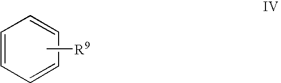

wherein R⁹ is defined as above. Conditions for the coupling are well known to the synthetic chemist. For example, reaction may take place in methanol under slightly acidic conditions, over up to 24 hours.

Compounds of formula III may be prepared by diazotisation of appropriate amines of formula V

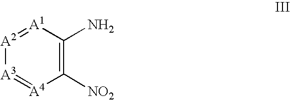

wherein A¹, A², A³, and A⁴ are defined as above. Methods of diazotisation are well known in the art, e.g. by reaction with NaNO₂/AcOH in an aqueous solution at 0 to 10° C.

Compounds of formula V may be synthesised by nitration, and subsequent deprotection, of a compound of formula VI,

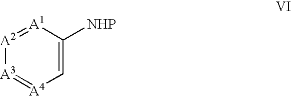

wherein A¹, A², A³, and A⁴ are as defined above and P represents a protecting group appropriate to the nitrating conditions. Nitration could be effected by, for example, cHNO₃/CH₂SO₄ in a solvent appropriate to the reaction conditions.

Compounds of formulas IV and VI may be made by conventional techniques known per se.

2-Phenylindazoles of formula I can be made by a variety of processes, as outlined in the scheme below.

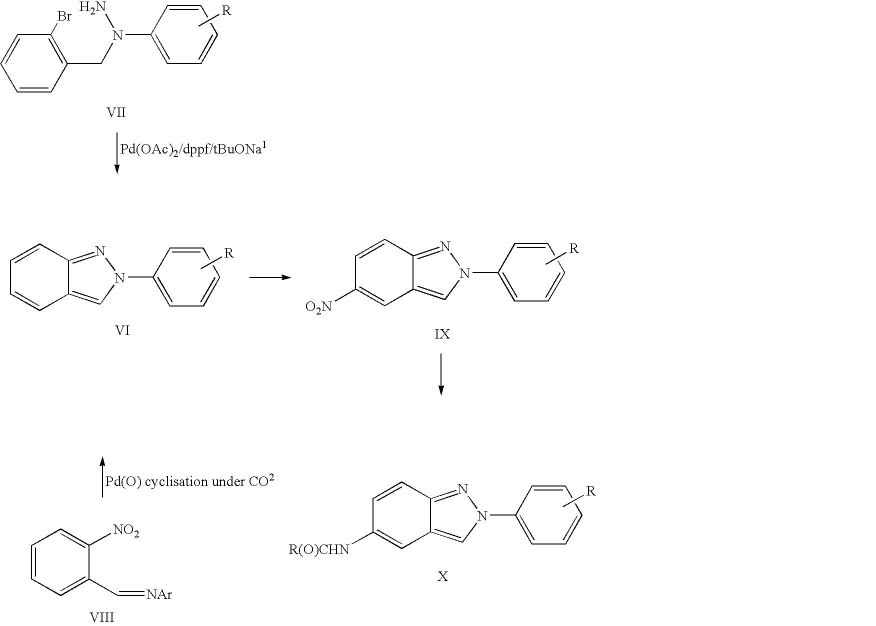

Phenyl indazoles may be made using known processes. For example hydrazines of formula VII may be cyclised using Pd (II) catalysis as described by Song, J. J. et al, Organic Letters, 2000, 2(4), 519-521.

Alternatively, phenyl indazoles of formula VII may be synthesised from an imine VIII using Pd (0) mediated cyclisation as described by Akazome, M. et al, J. Chem. Soc. Chemical Communications, 1991, 20, 1466-7.

The phenyl indazoles may then be manipulated using processes known to the skilled man. For example, nitration (as described by Elguero, J. et al, Bulletin des Societes Chimiques Belges, 1996, 105(6), 355-358) gives nitro compound IX. The skilled man is well aware of processes by which nitro compounds may be manipulated to give a wide range of functionality. For example, reduction of the nitro compound, for example using Sn/HCl, followed by acylation, for example using an acid chloride and triethyl amine in CH₂Cl₂ gives an amide X.

In the above processes it may be necessary for any functional groups, e.g. hydroxy or amino groups, present in the starting materials to be protected, thus it may be necessary to remove one or more protective groups to generate the compound of formula I.

Suitable protecting groups and methods for their removal are, for example, those described in “Protective Groups in Organic Synthesis” by T. Greene and P. G. M. Wutts, John Wiley and Sons Inc., 1991. Hydroxy groups may, for example, be protected by arylmethyl groups such as phenylmethyl, diphenylmethyl or triphenylmethyl; acyl groups such as acetyl, trichloroacetyl or trifluoroacetyl; or as tetrahydropyranyl derivatives. Suitable amino protecting groups include arylmethyl groups such as benzyl, (R,S)-α-phenylethyl, diphenylmethyl or triphenylmethyl, and acyl groups such as acetyl, trichloroacetyl or trifluoroacetyl. Conventional methods of deprotection may be used including hydrogenolysis, acid or base hydrolysis, or photolysis. Arylmethyl groups may, for example, be removed by hydrogenolysis in the presence of a metal catalyst e.g. palladium on charcoal. Tetrahydropyranyl groups may be cleaved by hydrolysis under acidic conditions. Acyl groups may be removed by hydrolysis with a base such as sodium hydroxide or potassium carbonate, or a group such as trichloroacetyl may be removed by reduction with, for example, zinc and acetic acid.

The compounds of formula I, and salts thereof, may be isolated from their reaction mixtures using conventional techniques.

Salts of the compounds of formula I may be formed by reacting the free acid, or a salt thereof, or the free base, or a salt or derivative thereof, with one or more equivalents of the appropriate base or acid. The reaction may be carried out in a solvent or medium in which the salt is insoluble or in a solvent in which the salt is soluble, e.g. ethanol, tetrahydrofuran or diethyl ether, which may be removed in vacuo, or by freeze drying. The reaction may also be a metathetical process or it may be carried out on an ion exchange resin.

Pharmaceutically acceptable salts of the compounds of formula I include alkali metal salts, e.g. sodium and potassium salts; alkaline earth metal salts, e.g. calcium and magnesium salts; salts of the Group III elements, e.g. aluminium salts; and ammonium salts. Salts with suitable organic bases, for example, salts with hydroxylamine; lower alkylamines, e.g. methylamine or ethylamine; with substituted lower alkylamines, e.g. hydroxy substituted alkylamines; or with monocyclic nitrogen heterocyclic compounds, e.g. piperidine or morpholine; and salts with amino acids, e.g. with arginine, lysine etc, or an N-alkyl derivative thereof; or with an aminosugar, e.g. N-methyl-D-glucamine or glucosamine. The non-toxic physiologically acceptable salts are preferred, although other salts are also useful, e.g. in isolating or purifying the product.

Diastereoisomers may be separated using conventional techniques, e.g. chromatography or fractional crystallisation. The various optical isomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation.

Substituents that alkyl may represent include methyl, ethyl, butyl, eg sec butyl.

Halogen may represent F, Cl, Br and I, especially Cl.

Examples of substituents that R³ in the compound of formula I may represent include alkyl, alkoxy or aryl, each optionally substituted by one or more, preferably one to three substituents, R₂, which may be the same or different.

In addition, compounds that may be mentioned include those of:

formula I of claim 1 or of formula II of claim 1 in which A⁵ represents N, wherein:

L is single bond and R³ represents:

thioalkyl optionally substituted by alkyl or optionally substituted aryl,

O-aryl or thioaryl, in which the aryl is optionally substituted,

optionally substituted aryl,

hydroxyl,

NR¹⁰R¹¹,

SO₂R¹²,

NR¹³SO₂R¹⁴,

C(═W)R¹⁶,

NR¹⁵C(═W)R¹⁷,

R¹⁰, R¹¹, R¹², R¹³, R¹⁴, R¹⁶ and R¹⁷, which may be the same or different, represent hydrogen, alkyl optionally substituted by optionally substituted aryl, optionally substituted aryl,

in addition,

R¹⁰ and R¹¹ together with the nitrogen to which they are attached may form a ring,

R¹² may have the same meaning as NR¹⁰R¹¹,

R¹⁶ and R¹⁷, which may be the same or different, may each represent

alkyl substituted by one or more of halogen, alkoxy optionally substituted aryl or optionally substituted aryl,

optionally substituted aryloxy,

aryl or NR¹⁰R¹¹,

and when R¹⁶ or R¹⁷ represents NR¹⁰R¹¹, one of R¹⁰ and R¹¹ may additionally represent CO alkyl optionally substituted or COaryl optionally substituted, and

in addition to the definitions shared with R¹⁷, R¹⁶ may represent hydroxyl;

or compounds of formula II of claim 1 in which A⁵ represents CH, and wherein

L is single bond and R³ represents:

thioalkyl optionally substituted by alkyl or optionally substituted aryl,

thioaryl, in which the aryl is optionally substituted,

optionally substituted aryl,

hydroxyl,

NO₂,

CN,

NR¹⁰R¹¹,

halogen,

SO₂R¹²,

NR¹³SO₂R¹⁴,

C(═W)R¹⁶,

OC(═W)NR¹⁰R¹¹

NR¹⁵C(═W)R¹⁷,

R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,

in addition,

R¹⁰ and R¹¹ together with the nitrogen to which they are attached may form a ring,

R¹² may have the same meaning as NR¹⁰R¹¹,

R¹⁶ and R¹⁷, which may be the same or different, may each represent

alkyl substituted by one or more of halogen, alkoxy optionally substituted aryl or optionally substituted aryl,

optionally substituted aryloxy,

aryl or NR¹⁰R¹¹,

and when R¹⁶ or R¹⁷ represents NR¹⁰R¹¹, one of R¹⁰ and R¹¹ may additionally represent CO alkyl optionally substituted or COaryl optionally substituted, and

in addition to the definitions shared with R¹⁷, R¹⁶ may represent hydroxyl.

Compounds that may be mentioned include those wherein R¹ and R², which may be the same or different, may represent:

alkyl optionally substituted by one or more halogen, alkoxy or optionally substituted aryl, thioaryl or aryloxy,

alkoxy optionally substituted by optionally by alkyl or optionally substituted aryl,

hydroxyl,

OC(═W)NR¹⁰R¹¹

aryl,

thioalkyl optionally substituted by alkyl or optionally substituted aryl,

thioaryl, in which the aryl is optionally substituted,

NO₂,

CN,

NR¹⁰R¹¹,

halogen,

SO₂R¹²,

NR¹³SO₂R¹⁴,

C(═W)R¹⁶,

NR¹⁵C(═W)R¹⁷,

P(═O)OR⁴⁰R⁴¹,

R₁₀, R₁₁, R₁₂, R₁₃, R₁₄, R₁₅, R₁₆, R₁₇, R₄₀ and R₄₁, which may be the same or different, represent hydrogen, alkyl optionally substituted by optionally substituted aryl, optionally substituted aryl,

in addition,

NR¹⁰R¹¹ together with the nitrogen to which they are attached may form a ring,

R¹² may have the same meaning as NR¹⁰R¹¹,

when R₁₇ represents NR¹⁰R¹¹, that NR¹⁰R¹¹ may represent hydrogen, COalkyl and CO optionally substituted aryl,

R¹⁶ may represent hydroxy, alkoxy, or NR¹⁰R¹¹,

and R¹⁷ may represent alkyl substituted by one or more of halogen, alkoxy, optionally substituted aryl or NR¹⁰R¹¹.

Other compounds that may be mentioned include those of either:

formula I of claim 1 or of formula II of claim 1 in which A⁵ represents N,

wherein:

L represents a linker group which is:

O, S or NR¹⁸,

alkylene, alkenylene, alkynylene, each of which may be optionally interrupted by one or more of O, S, NR¹⁸, or one or more C—C single, double or triple bonds,

and R¹⁸ represents hydrogen, alkyl, COR¹⁶.

or a compound of formula II of claim 1 in which A⁵ represents CH, wherein:

L represents a linker group which is:

O, S, NR⁸,

alkylene, alkenylene, alkynylene, each of which may be optionally interrupted by one or more of O, S, NR¹⁸, or one or more C—C single, double or triple bonds,

a —N—N— single or double bond,

and R¹⁸ represents hydrogen, alkyl, COR¹⁶.

Alkyl may represent any alkyl chain. Alkyl includes straight and branched, saturated and unsaturated alkyl, as well as cyclic alkyl, such as cyclopropyl, cyclobutyl, cyclopentyl, cyclohexyl and cycloheptyl. However, preferably, when any of the substituents represents alkyl, alkyl is saturated, linear or branched and has from 1 to 10 carbon atoms, preferably from 1 to 8 carbon atoms and more preferably from 1 to 6 carbon atoms. When any of the substituents represents alkyl, a particularly preferred group is cycloalkyl, for example cyclopropyl, cyclobutyl, cyclopentyl, cyclohexyl and cycloheptyl.

Aryl may represent any aromatic system. Preferably, in the compounds of formula I, aryl is an aromatic hydrocarbon or a 5 to 10 membered aromatic heterocycle containing 1 to 4 hetero atoms selected from an oxygen atom, a sulphur atom and a nitrogen atom as a ring constituent besides carbon. We prefer heterocycles which contain one or two heteroatoms. Aromatic heterocycles that may be mentioned include furan, thiophene, pyrrole, and pyridine.

Particularly preferably, when aryl is an aromatic hydrocarbon, aryl represents a 6 to 10 membered monocyclic or bicyclic system, for example phenyl or naphthalene.

Saturated and unsaturated heterocycles that may be mentioned include those containing 4 to 7 ring atoms, preferably 5 or 6 ring atoms, preferably containing one to two heteroatoms selected from N, S and O. Heterocycles that may be mentioned include pyrrolidine, piperidine, tetrahydrofuran, piperazine and morpholine. N-containing heterocycles are particularly preferred, eg when NR¹⁰R¹¹ forms a heterocyclic ring.

As detailed above, when an adjacent pair of A¹-A⁴ each represent CR¹, the adjacent carbon atoms, together with their substituents may form a ring B. Also, when A⁵ represents CR¹, then A⁵ and CR¹ together with their substituents may form a ring C. Preferably ring B and/or ring C is a saturated or unsaturated 3 to 10 membered carbocylic or heterocyclic ring.

Particularly preferably ring B is benzene ring.

Particularly preferably ring C is a 3-10 membered saturated or unsaturated heterocyclic ring.

We particularly prefer compounds in which at least one R₁ represents NR¹⁵C(═W)R¹⁷, most particularly the group NR¹⁵COR¹⁷.

We also prefer compounds in which at least one R¹ represents CONR¹⁰R¹¹.

For one group of particularly preferred compounds at least one R₁ represents an amide group NHCOR¹⁷, wherein R¹⁷ is selected from:

alkyl C₁-C₆,

alkyl C₁-C₆ substituted by phenyl

alkyl C₁-C₆ substituted by alkoxy C₁-C₆,

haloalkyl C₁-C₆,

perfluoroalkyl C₁-C₆,

phenyl optionally substituted by one or more of halogen, alkyl C₁-C₆, alkoxy C₁-C₆, amino, (alkyl C₁-C₆)amino, di(alkyl C₁-C₆)amino or phenyl,

CH:CH phenyl,

naphthyl, pyridinyl, thiophenyl and furanyl.

We prefer compounds in which one or both of R¹ and R² are other than —COOH.

For another group of particularly preferred compounds at least one R¹ represents a group NR¹⁵CONR¹⁰R¹¹, then in which R¹⁰ and R¹¹, which may be the same or different, are selected from optionally substituted aryl, alkyl and COaryl optionally substituted. A particularly preferred group which at least one of R¹ may represent is NHCONHR¹⁵ and R¹⁵ is selected from phenyl, alkyl C₁ to C₆ and COphenyl optionally substituted by one or more halogen.

For another group of particularly preferred compounds at least one R¹ represents alkyl C₁ to C₆, optionally substituted by phenyl or a 5 or 6-membered saturated or unsaturated heterocycle containing one to two heteroatoms selected from N, S and O. Preferred heterocycles include thiophene, furan, pyridine and pyrrole.

For another group of particularly preferred compounds at least one R¹ represents COR¹⁶ and R¹⁶ is alkoxy C₁-C₆, amino, (alkyl C₁-C₆)amino or di(alkyl C₁-C₆)amino.

For another group of particularly preferred compounds at least one R₁ represents:

NO₂,

halogen,

amino or (alkyl C₁-C₆)amino or di(alkyl C₁-C₆)amino in which the alkyl C₁ to C₆ is optionally substituted by phenyl or a 5 or 6 membered saturated or unsaturated heterocycle,

NHSO₂alkyl C₁-C₆, NHSO₂phenyl,

SO₂alkyl C₁-C₆,

phenyl optionally substituted by C₁ to C₆ alkoxy C₁-C₆,

a 5-10 membered, saturated or unsaturated, mono- or bi-cyclic heterocycle containing from 1-3 heteroatoms selected from N, S and O.

There is also wide scope for variation of the group R³. Preferably R³ represents aryl and is optionally substituted by one to three substituents, R², which may be the same or different.

Particularly preferably, R³ is a 5-10 membered aromatic mono- or bi-cyclic system, especially a hydrocarbon 5-10 membered aromatic mono- or bi-cyclic system, for example benzene or naphthalene.

Alternatively, the 5-10 membered aromatic mono- or bi-cyclic system, may be a heterocyclic system containing up to three heteroatoms selected from N, O and S, for example a thiophene, furan, pyridine or pyrrole.

Preferably the substituent(s) R² is/are selected from:

alkyl C₁-C₆, optionally substituted by thiophenyl or phenoxy, each optionally substituted by halogen,

alkoxy C₁-C₆

phenyl,

thioalkyl C₁-C₆

thiophenyl, optionally substituted by halogen,

NO₂,

CN

NR¹⁰R¹¹, in which R¹⁰ and R¹¹, which may be the same or different represent hydrogen, alkyl C₁-C₆, or together with the nitrogen to which they are attached form a 5 to 7 membered ring which may contain one or more additional heteroatoms selected from N, O and S,

halogen

SO₂R¹², in which R¹² represents a 5 to 7 membered ring which may contain one or more additional heteroatoms selected from N, O and S

NHCOR¹⁷, in which R¹⁷ represents
alkyl C₁-C₆, optionally substituted by:
   phenyl or halogen, or
   phenyl optionally substituted by alkoxy C₁-C₆, carboxy or
   halogen, or
   a 5 or 6 membered saturated or unsaturated heterocycle,
phenyl or a 5 or 6 membered saturated or unsaturated heterocycle optionally substituted by halogen, alkoxy C₁ to C₆, carboxy or a group SO₂NR¹⁰R¹¹,

Particularly preferably when R² represents NR¹⁰R¹¹, NR¹⁰R¹¹ represents N-pyrrole, N-piperidine, N′(C₁-C₆) alkyl N piperazine or N-morpholine.

Preferably the linker group L represents:

—NH.NH—

—CH═CH—,

—C≡C—, or

—NCOR¹⁶ in which R¹⁶ represents phenyl or a 5 or 6 membered saturated or unsaturated heterocycle optionally substituted by halogen, alkoxy C₁ to C₆, carboxy.

A¹-A⁴ may represent N or CR¹. Consequently, the six membered ring may contain 1, 2, 3 or 4 nitrogen atoms. Embodiments of the invention exist in which two of A¹-A⁴ represent nitrogen, one of A¹-A⁴ represents nitrogen and in which all of A¹-A⁴ represents CR¹.

In a particularly preferred group of compounds:

A¹, A², A³, A⁴ and A⁵ which may be the same or different, represent N or CR¹,
R⁹ represents -L-R³, in which L is a single bond or a linker group,

either the compound is of formula I or of formula II wherein A⁵ represents N,

and

L is single bond and R³ represents:

thioalkyl optionally substituted by alkyl or optionally substituted aryl,

thioaryl, in which the aryl is optionally substituted,

optionally substituted aryl,

hydroxyl,

NR¹⁰R¹¹,

SO₂R¹²,

NR¹³SO₂R¹⁴,

C(═W)R¹⁶,

NR¹⁵C(═W)R¹⁷,

R¹⁰, R¹¹, R¹², R¹³, R¹⁴, R¹⁶ and R¹⁷, which may be the same or different, represent hydrogen, alkyl optionally substituted by optionally substituted aryl, optionally substituted aryl,

in addition,

R¹⁰ and R¹¹ together with the nitrogen to which they are attached may form a ring,

R¹² may have the same meaning as NR¹⁰R¹¹,

R¹⁶ and R¹⁷, which may be the same or different, may each represent

alkyl substituted by one or more of halogen, alkoxy optionally substituted aryl or optionally substituted aryl,

optionally substituted aryloxy,

aryl or NR¹⁰R¹¹,

and when R¹⁶ or R¹⁷ represents NR¹⁰R¹¹, one of R¹⁰ and R¹¹ may additionally represent CO alkyl optionally substituted or COaryl optionally substituted, and

in addition to the definitions shared with R¹⁷, R¹⁶ may represent hydroxyl;

or the compound is of formula II in which A⁵ represents CH, and wherein L is

single bond and R³ represents:

thioalkyl optionally substituted by alkyl or optionally substituted aryl,

thioaryl, in which the aryl is optionally substituted,

optionally substituted aryl,

hydroxyl,

NO₂,

CN,

NR¹⁰R¹¹,

halogen,

SO₂R¹²,

NR¹³SO₂R¹⁴,

C(═W)R¹⁶,

OC(═W)NR¹⁰R¹¹

NR¹⁵C(═W)R¹⁷,

R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,

in addition,

R¹⁰ and R¹¹ together with the nitrogen to which they are attached may form a ring,

R¹² may have the same meaning as NR¹⁰R¹¹,

R¹⁶ and R¹⁷, which may be the same or different, may each represent

alkyl substituted by one or more of halogen, alkoxy optionally substituted aryl or optionally substituted aryl,

optionally substituted aryloxy,

aryl or NR¹⁰R¹¹,

and when R¹⁶ or R¹⁷ represents NR¹⁰R¹¹, one of R¹⁰ and R¹¹ may additionally represent CO alkyl optionally substituted or COaryl optionally substituted, and in addition to the definitions shared with R¹⁷, R¹⁶ may represent hydroxyl and in addition,

R¹ and R², which may be the same or different, represent:

alkyl optionally substituted by one or more halogen, alkoxy or optionally substituted aryl, thioaryl or aryloxy,

alkoxy optionally substituted by optionally by alkyl or optionally substituted aryl,

hydroxyl,

OC(═W)NR¹⁰R¹¹

aryl,

thioalkyl optionally substituted by alkyl or optionally substituted aryl,

thioaryl, in which the aryl is optionally substituted,

NO₂,

CN,

NR¹⁰R¹¹,

halogen,

SO₂R¹²,

NR¹³SO₂R¹⁷,

C(═W)R¹⁶,

NR¹⁵C(═W)R¹⁷,

R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,

in addition,

NR¹⁰R¹¹ together with the nitrogen to which they are attached may form a ring,

R¹² may have the same meaning as NR¹⁰R¹¹,

when R₁₇ represents NR¹⁰R¹¹, that NR¹⁰R¹¹ may represent hydrogen, COalkyl and CO optionally substituted aryl,

R¹⁶ may represent hydroxy, alkoxy, or NR¹⁰R¹¹,

and R¹⁷ may represent alkyl substituted by one or more of halogen, alkoxy, optionally substituted aryl or NR¹⁰R¹¹.

when an adjacent pair of A₁-A₄ each represent CR₁, then the adjacent carbon atoms, together with their substituents may form a ring B,
or a pharmaceutically acceptable salt thereof,
in the manufacture of a medicament for the therapeutic and/or prophylactic treatment of Duchenne muscular dystrophy, Becker muscular dystrophy or cachexia.
We also provide a method for the treatment or prophylaxis of Duchenne muscular dystrophy, Becker muscular dystrophy or cachexia in a patient in need thereof, comprising administering to the patient an effective amount of a compound of formula (I) or (II) or a pharmaceutical acceptable salt.

The compounds of formula I for use in the treatment of DMD will generally be administered in the form of a pharmaceutical composition.

Thus, according to a further aspect of the invention there is provided a pharmaceutical composition including preferably less than 80% w/w, more preferably less than 50% w/w, e.g. 0.1 to 20%, of a compound of formula I, or a pharmaceutically acceptable salt thereof, as defined above, in admixture with a pharmaceutically acceptable diluent or carrier.

We also provide a process for the production of such a pharmaceutical composition which comprises mixing the ingredients. Examples of pharmaceutical formulations which may be used, and suitable diluents or carriers, are as follows:

for intravenous injection or infusion—purified water or saline solution;

for inhalation compositions—coarse lactose;

for tablets, capsules and dragees—microcrystalline cellulose, calcium phosphate, diatomaceous earth, a sugar such as lactose, dextrose or mannitol, talc, stearic acid, starch, sodium bicarbonate and/or gelatin;

for suppositories—natural or hardened oils or waxes.

When the compound is to be used in aqueous solution, e.g. for infusion, it may be necessary to incorporate other excipients. In particular there may be mentioned chelating or sequestering agents, antioxidants, tonicity adjusting agents, pH-modifying agents and buffering agents.

Solutions containing a compound of formula I may, if desired, be evaporated, e.g. by freeze drying or spray drying, to give a solid composition, which may be reconstituted prior to use.

When not in solution, the compound of formula I preferably is in a form having a mass median diameter of from 0.01 to 10 μm. The compositions may also contain suitable preserving, stabilising and wetting agents, solubilisers, e.g. a water-soluble cellulose polymer such as hydroxypropyl methylcellulose, or a water-soluble glycol such as propylene glycol, sweetening and colouring agents and flavourings. Where appropriate, the compositions may be formulated in sustained release form.

The content of compound formula I in a pharmaceutical composition is generally about 0.01-about 99.9 wt %, preferably about 0.1-about 50 wt %, relative to the entire preparation.

The dose of the compound of formula I is determined in consideration of age, body weight, general health condition, diet, administration time, administration method, clearance rate, combination of drugs, the level of disease for which the patient is under treatment then, and other factors.

While the dose varies depending on the target disease, condition, subject of administration, administration method and the like, for oral administration as a therapeutic agent for the treatment of Duchenne muscular dystrophy in a patient suffering from such a disease is from 0.01 mg-10 g, preferably 0.1-100 mg, is preferably administered in a single dose or in 2 or 3 portions per day.

The potential activity of the compounds of formula I for use in the treatment of DMD may be demonstrated in the following predictive assay and screens.

1. Luciferase Reporter Assay (Murine H2K Cells)

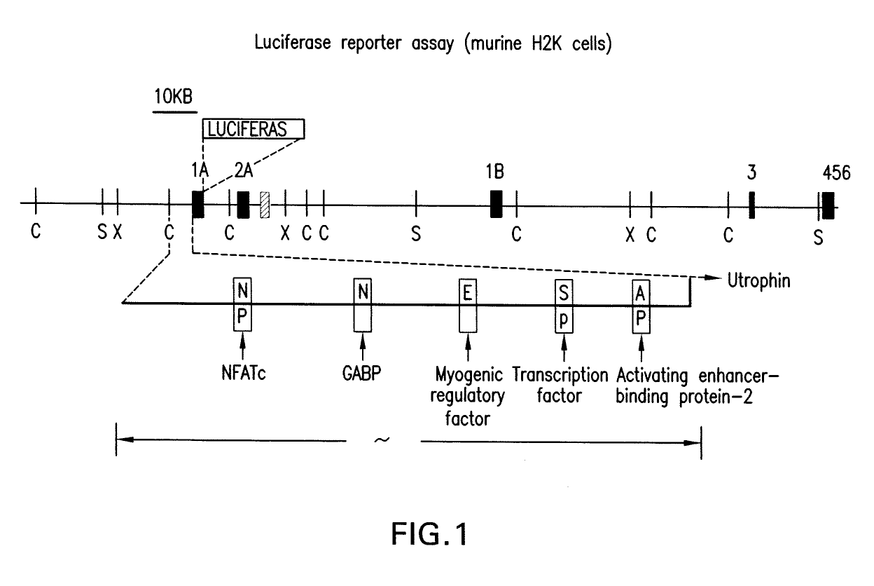

The cell line used for the screen is an immortalized mdx mouse H2K cell line that has been stably transfected with a plasmid containing ≈5 kb fragment of the Utrophin A promoter including the first untranslated exon linked to a luciferase reporter gene (see FIG. 1).

Under conditions of low temperature and interferon containing media, the cells remain as myoblasts. These are plated into 96 well plates and cultured in the presence of compound for three days. The level of luciferase is then determined by cell lysis and reading of the light output from the expressed luciferase gene utilising a plate luminometer.

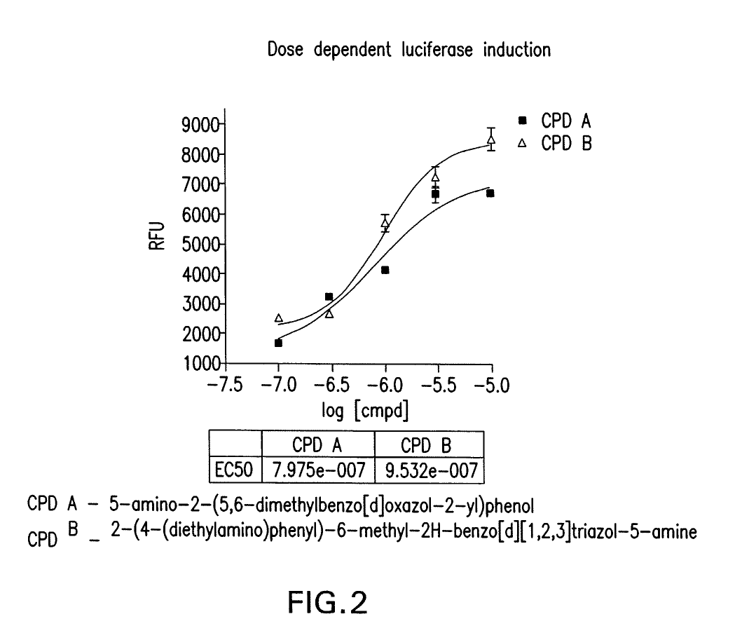

Example of pharmacological dose response of compounds in the assay is shown in FIG. 2

2. mdx Mouse

Data obtained from the ADMET data was prioritised and the compounds with the best in vitro luciferase activity and reasonable ADMET data were prioritised for testing in the mdx proof of concept study where the outcome was to identify whether any of the compounds had the ability to increase the levels of utrophin protein in dystrophin deficient muscle when compared to vehicle only dosed control animals.

There were two animals injected with 10 mg/kg of compound administered ip daily for 28 days plus age matched controls. Muscle samples were taken and processed for sectioning (to identify increases in sarcolemmal staining of utrophin) and Western blotting (to identify overall increases in utrophin levels).

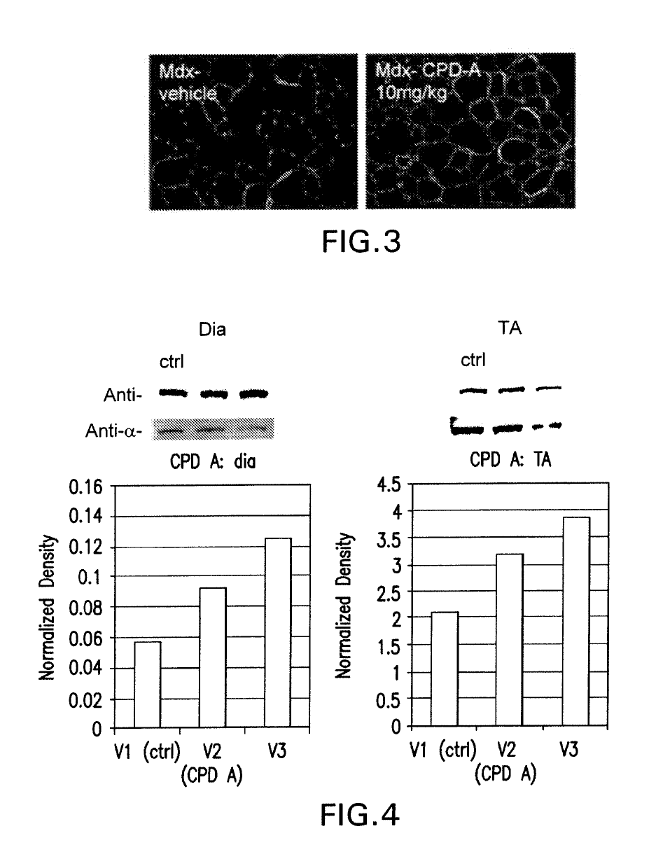

FIG. 3. shows an example of TA muscle sections stained with antibody specific for mouse utrophin. Comparison to the mdx muscle only injected with vehicle shows an increase in the amount of sarcolemmal bound utrophin.

Muscles from the above treated mice were also excised and processed for Western blotting and stained with specific antibodies (see FIG. 4). Again using muscle dosed with CPD-A shows a significant increase in the overall levels of utrophin present in both the TA leg muscle and the diaphragm. Both mice exposed to CPD-A (V2 and V3) showed increased levels of utrophin expression compared to control.

Positive upregulation data from the first 28 day study were then repeated in a further two mouse 28 day study. A total of three different compounds have shown in duplicate the ability to increase the level of utrophin expression in the mdx mouse when delivered daily by ip for 28 days. This data demonstrates the ability of the compound when delivered ip causes a significant increase in the levels of utrophin found in the mdx muscle and therefore gives us the confidence that this approach will ameliorate the disease as all the published data to date demonstrates that any increase of utrophin levels over three fold has significant functional effects on dystrophin deficient muscle.

The H2K/mdx/Utro A reporter cell line maintenance

The H2K/mdx/Utro A reporter cell line was passaged twice a week until ≦30% confluent. The cells were grown at 33° C. in the presence of 10% CO₂

To remove the myoblasts for platting, they were incubated with Trypsin/EDTA until the monolayer started to detach.

Growth Medium
DMEM Gibco 41966
20% FCS
1% Pen/Strep
1% glutamine
10 mls Chick embryo extract
Interferon (1276 905 Roche) Add fresh 10 μl/50 mls medium

Luciferase Assay for 96 Well Plates

The H2K/mdx/Utro A reporter cell line cells were plated out into 96 well plates (Falcon 353296, white opaque) at a density of approximately 5000 cells/well in 190 μl normal growth medium. The plates were then incubated at 33° C. in the presence of 10% CO₂ for 24 hrs.

Compounds were dosed by adding 10 μl of diluted compound to each well giving a final concentration of 10 μM. The plates were then incubated for a further 48 hrs

Cells were then lysed in situ following the manufacture's protocols (Promega Steady-Glo Luciferase Assay System (E2520). Then counted for 10 seconds using a plate luminometer (Victorl420).

Compound Storage

Compounds for screening were stored at −20° C. as 10 mM stocks in 100% DMSO until required.

Injection of mdx Mice with Compounds

Mdx from a breeding colony were selected for testing. Mice were injected daily with either vehicle or 10 mg/kg of compound using the intreperitoneal route (ip). Mice were weighed and compounds diluted in 5% DMSO, 0.1% tween in PBS.

Mice were sacrificed by cervical dislocation at desired time points, and muscles excised for analysis

Muscle Analysis

Immunohistochemistry

Tissues for sectioning were dissected, immersed in OCT (Bright Cryo-M-Bed) and frozen on liquid nitrogen cooled isopentane. Unfixed 8 μM cryosections were cut on a Bright Cryostat, and stored at −80° C.

In readiness for staining, sections were blocked in 5% foetal calf serum in PBS for 30 mins. The primary antibodies were diluted in blocking reagent and incubated on sections for 1.5 hrs in a humid chamber then washed three times for 5 mins in PBS. Secondary antibodies also diluted in blocking reagent, were incubated for 1 hr in the dark in a humid chamber. Finally sections were washed three times 5 mins in PBS and coverslip Mounted with hydromount. Slides were analysed using a Leica fluorescent microscope.

Results

Biological activity as assessed using the luciferase reporter assay in murine H2K cells, and is classified as follows:

+ Up to 200% relative to control

++ Between 201% and 300% relative to control

+++ Between 301% and 400% relative to control

++++ Above 401% relative to control

TABLE 1
Compounds made by methods described herein

| Example | | |
|---|---|---|
| number | Chemical Name | Activity |
| 1 | N-(2-(4-chlorophenyl)-6-methyl-2H-benzo[d][1,2,3]triazol-5- | +++ |
| | yl)nicotinamide | |
| 2 | N-(2-(4-chlorophenyl)-6-methyl-2H-benzo[d][1,2,3]triazol-5- | ++ |
| | yl)isonicotinamide | |
| 3 | N-(2-(4-chlorophenyl)-6-methyl-2H-benzo[d][1,2,3]triazol-5- | + |
| | yl)benzamide | |
| 4 | N-(2-(4-chlorophenyl)-6-methyl-2H-benzo[d][1,2,3]triazol-5-yl)-4- | ++ |
| | methoxybenzamide | |
| 5 | N-(2-(4-chlorophenyl)-6-methyl-2H-benzo[d][1,2,3]triazol-5-yl)-2- | + |
| | methoxybenzamide | |
| 6 | N-(2-(4-chlorophenyl)-6-methyl-2H-benzo[d][1,2,3]triazol-5- | +++ |
| | yl)thiophene-2-carboxamide | |
| 7 | N-(2-(4-chlorophenyl)-6-methyl-2H-benzo[d][1,2,3]triazol-5- | + |
| | yl)propionamide | |
| 8 | N-(2-(4-chlorophenyl)-6-methyl-2H-benzo[d][1,2,3]triazol-5- | ++ |
| | yl)butyramide | |
| 9 | N-(2-(4-chlorophenyl)-6-methyl-2H-benzo[d][1,2,3]triazol-5- | ++ |
| | yl)pentanamide | |
| 10 | N-(2-(4-chlorophenyl)-6-methyl-2H-benzo[d][1,2,3]triazol-5- | ++ |
| | yl)isobutyramide | |
| 11 | N-(2-(4-chlorophenyl)-6-methyl-2H-benzo[d][1,2,3]triazol-5-yl)furan-2- | ++ |
| | carboxamide | |
| 12 | N-(2-(4-(diethylamino)phenyl)-6-methyl-2H-benzo[d][1,2,3]triazol-5- | +++ |
| | yl)nicotinamide | |
| 13 | N-(2-(4-(diethylamino)phenyl)-6-methyl-2H-benzo[d][1,2,3]triazol-5- | +++ |
| | yl)isonicotinamide | |
| 14 | N-(2-(4-(diethylamino)phenyl)-6-methyl-2H-benzo[d][1,2,3]triazol-5- | + |
| | yl)propionamide | |
| 15 | N-(2-(4-(diethylamino)phenyl)-6-methyl-2H-benzo[d][1,2,3]triazol-5- | + |
| | yl)butyramide | |
| 16 | N-(2-(4-(diethylamino)phenyl)-6-methyl-2H-benzo[d][1,2,3]triazol-5- | + |
| | yl)pentanamide | |
| 17 | N-(2-(4-(diethylamino)phenyl)-6-methyl-2H-benzo[d][1,2,3]triazol-5- | + |
| | yl)isobutyramide | |
| 18 | N-(2-(4-(diethylamino)phenyl)-6-methyl-2H-benzo[d][1,2,3]triazol-5- | + |
| | yl)furan-2-carboxamide | |
| 19 | 2-(4-(diethylamino)phenyl)-2H-benzo[d][1,2,3]triazol-5-amine | +++ |
| 20 | N-(2-(4-(diethylamino)phenyl)-2H-benzo[d][1,2,3]triazol-5- | ++ |
| | yl)nicotinamide | |
| 21 | N-(2-(4-(diethylamino)phenyl)-2H-benzo[d][1,2,3]triazol-5- | + |
| | yl)isonicotinamide | |
| 22 | N-(2-(4-(diethylamino)phenyl)-2H-benzo[d][1,2,3]triazol-5-yl)acetamide | ++ |
| 23 | N-(2-(4-(diethylamino)phenyl)-2H-benzo[d][1,2,3]triazol-5- | + |
| | yl)propionamide | |
| 24 | N-(2-(4-(diethylamino)phenyl)-2H-benzo[d][1,2,3]triazol-5- | + |
| | yl)butyramide | |
| 25 | N-(2-(4-(diethylamino)phenyl)-2H-benzo[d][1,2,3]triazol-5- | + |
| | yl)pentanamide | |
| 26 | N-(2-(4-(diethylamino)phenyl)-2H-benzo[d][1,2,3]triazol-5- | + |
| | yl)isobutyramide | |
| 27 | N-(2-(4-(diethylamino)phenyl)-2H-benzo[d][1,2,3]triazol-5-yl)furan-2- | + |
| | carboxamide | |
| 28 | N-(2-(4-(diethylamino)phenyl)-2H-benzo[d][1,2,3]triazol-5- | + |
| | yl)thiophene-2-carboxamide | |
| 29 | N-(2-(4-(diethylamino)phenyl)-2H-benzo[d][1,2,3]triazol-5- | + |
| | yl)benzamide | |
| 30 | N-(2-(4-(diethylamino)phenyl)-2H-benzo[d][1,2,3]triazol-5-yl)-4- | + |
| | methoxybenzamide | |
| 31 | N-(2-(4-(diethylamino)phenyl)-2H-benzo[d][1,2,3]triazol-5-yl)-2- | + |
| | methoxybenzamide | |
| 32 | 4-chloro-N-(2-(4-(diethylamino)phenyl)-2H-benzo[d][1,2,3]triazol-5- | + |
| | yl)benzamide | |
| 33 | N-(2-(4-(diethylamino)phenyl)-2H-benzo[d][1,2,3]triazol-5-yl)-4- | + |
| | (dimethylamino)benzamide | |
| 34 | 6-methyl-2-(4-morpholinophenyl)-2H-benzo[d][1,2,3]triazol-5-amine | ++ |
| 35 | N-(2-(4-chlorophenyl)-2H-benzo[d][1,2,3]triazol-5-yl)propionamide | ++++ |
| 36 | N-(2-(4-chlorophenyl)-2H-benzo[d][1,2,3]triazol-5-yl)butyramide | +++ |
| 37 | N-(2-(4-chlorophenyl)-2H-benzo[d][1,2,3]triazol-5-yl)isobutyramide | ++++ |
| 38 | 2-(4-chlorophenyl)-2H-benzo[d][1,2,3]triazol-5-amine | + |
| 39 | N-(2-(4-chlorophenyl)-2H-benzo[d][1,2,3]triazol-5-yl)acetamide | ++++ |
| 40 | 2-(4-(piperidin-1-yl)phenyl)-2H-benzo[d][1,2,3]triazol-5-amine | ++ |
| 41 | 2-(4-(dimethylamino)phenyl)-2H-benzo[d][1,2,3]triazol-5-amine | +++ |
| 42 | 2-(4-(4-methylpiperazin-1-yl)phenyl)-2H-benzo[d][1,2,3]triazol-5-amine | + |
| 43 | 2-(4-chlorophenyl)-6-methyl-2H-benzo[d][1,2,3]triazol-5-amine | ++++ |
| 44 | 2-(4-chlorophenyl)-6-(methylsulfonyl)-2H-indazole | ++ |
| 45 | 2-(4-chlorophenyl)-6-nitro-2H-indazole | + |
| 46 | N-(2-(4-chlorophenyl)-2H-indazol-6-yl)isobutyramide | ++++ |
| 47 | 2-(4-chlorophenyl)-6-(methylsulfonyl)-2H-benzo[d][1,2,3]triazole 1- | + |
| | oxide | |
| 48 | 2-(4-chlorophenyl)-2H-indazole | ++++ |
| 49 | 2-(4-chlorophenyl)-5-(methylsulfonyl)-2H-benzo[d][1,2,3]triazole | + |
| 50 | 2-(3,4-dichlorophenyl)-5-(methylsulfonyl)-2H-benzo[d][1,2,3]triazole | ++ |
| 51 | 2-(3′,4′-dichlorophenyl)-5-(ethylsulfonyl)-benzotriazole | +++ |
| 52 | 2-(4′-chlorophenyl)-5-(ethylsulfonyl)-benzotriazole | ++++ |
| 53 | N-(2-(3,4-Dichlorophenyl)-2H-benzo[d][1,2,3]triazol-5-yl)isobutyramide | ++++ |
| 54 | 6-(Methylsulfonyl)-2-(naphthalen-2-yl)-2H-benzo[d][1,2,3]triazole 1- | ++ |
| | oxide | |
| 55 | 5-(Methylsulfonyl)-2-(naphthalen-2-yl)-2H-benzo[d][1,2,3]triazole | ++++ |
| 56 | 2-(4′-Chlorophenyl)-6-(isopropylsulfonyl)-2H-indazole | ++++ |

TABLE 2
Compounds made by analogues methods to those
described herein, or by literature methods known or
adapted by the persons skilled in the art.

| Example | | |
|---|---|---|
| number | Chemical Name | Activity |
| 57 | 5-nitro-2-phenyl-2H-benzo[d][1,2,3]triazole | ++ |
| 58 | 2-p-tolyl-2H-benzo[d][1,2,3]triazol-5-amine | + |
| 59 | 2-(4-nitrophenyl)-2H-benzo[d][1,2,3]triazol-5-amine | ++ |
| 60 | 2-(4-methoxyphenyl)-2H-benzo[d][1,2,3]triazol- | + |
| | 5-amine | |
| 61 | 2-(3-chlorophenyl)-2H-benzo[d][1,2,3]triazol- | + |
| | 5-amine | |
| 62 | 2-phenyl-2H-benzo[d][1,2,3]triazol-5-amine | ++ |
| 63 | 2-(3,4-dimethylphenyl)-2H-benzo[d][1,2,3]triazol- | + |
| | 5-amine | |
| 64 | 2-(4-ethoxyphenyl)-6-methyl-2H- | ++ |
| | benzo[d][1,2,3]triazol-5-amine | |
| 65 | 6-methyl-2-p-tolyl-2H-benzo[d][1,2,3]triazol-5- | ++ |
| | amine | |
| 66 | N-(2-(4-methoxyphenyl)-6-methyl-2H- | + |
| | benzo[d][1,2,3]triazol-5-yl)acetamide | |
| 67 | N-(6-methyl-2-phenyl-2H-benzo[d][1,2,3]triazol- | ++ |
| | 5-yl)acetamide | |
| 68 | 2-(4-ethylphenyl)-2H-benzo[d][1,2,3]triazol-5-amine | ++ |
| 69 | N-(2-(4-fluorophenyl)-2H-benzo[d][1,2,3]triazol-5- | ++ |
| | yl)acetamide | |
| 57 | N-(2-(4-Chlorophenyl)-6-methyl-2H- | +++ |
| | benzo[d][1,2,3]triazol-5-yl)acetamide | |
| 58 | 2-(4-Fluorophenyl)-2H-benzo[d][1,2,3]triazol-5- | +++ |
| | amine | |
| 59 | 2-(4-(Diethylamino)phenyl)-6-methyl-2H- | ++++ |
| | benzo[d][1,2,3]triazol-5-amine | |
| 60 | 2-(5-Amino-2H-benzo[d][1,2,3]triazol-2-yl)phenol | ++++ |
| 61 | 6-Methyl-2-p-tolyl-2H-benzo[d][1,2,3]triazol-5- | ++ |
| | amine | |
| 62 | 6-Methyl-2-phenyl-2H-benzo[d][1,2,3]triazol-5- | ++ |
| | amine | |

### EXPERIMENTAL

HPLC-UV-MS was performed on a Gilson 321 HPLC with detection performed by a Gilson 170 DAD and a Finnigan AQA mass spectrometer operating in electrospray ionisation mode. The HPLC column used is a Phenomenex Gemini C18 150×4.6 mm.

Preparative HPLC was performed on a Gilson 321 with detection performed by a Gilson 170 DAD. Fractions were collected using a Gilson 215 fraction collector. The preparative HPLC column used is a Phenomenex Gemini C18 150×10 mm and the mobile phase is acetonitrile/water.

¹H NMR spectra were recorded on a Bruker instrument operating at 300 MHz. NMR spectra were obtained as CDCl₃ solutions (reported in ppm), using chloroform as the reference standard (7.25 ppm) or DMSO-D₆ (2.50 ppm). When peak multiplicities are reported, the following abbreviations are used s (singlet), d (doublet), t (triplet), m (multiplet), br (broadened), dd (doublet of doublets), dt (doublet of triplets), td (triplet of doublets). Coupling constants, when given, are reported in Hertz (1 Hz).

Column chromatography was performed either by flash chromatography (40-65 μm silica gel) or using an automated purification system (SP1™ Purification System from Biotage®). Reactions in the microwave were done in an Initiator 8™ (Biotage).

The abbreviations used are DMSO (dimethylsulfoxide), HCl (hydrochloric acid), MgSO₄ (magnesium sulfate), NaOH (sodium hydroxide), Na₂CO₃ (sodium carbonate), NaHCO₃ (sodium bicarbonate), THF (tetrahydrofuran).

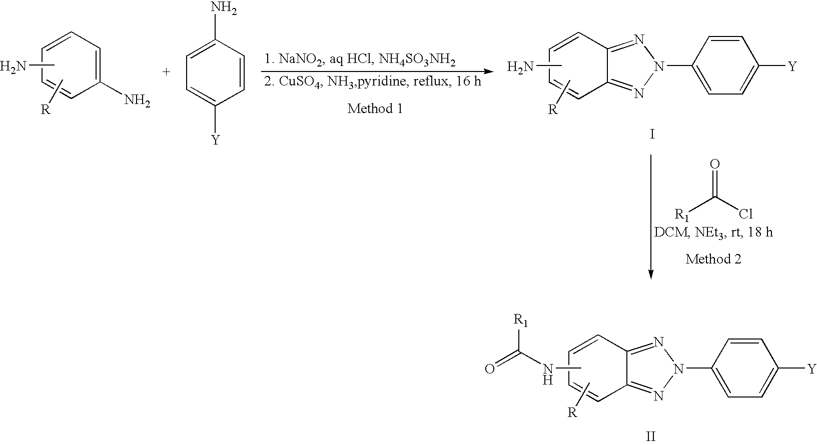

Method 1: Compounds I

### 2-(4-(Diethylamino)phenyl)-2H-benzo[d][1,2,3]triazol-5-amine

An aqueous solution (10 mL) of sodium nitrite (764 mg, 11.1 mmol) was added dropwise to a solution of N,N-diethyl-p-phenylenediamine (1.54 mL, 9.3 mmol) in 10% aqueous hydrochloric acid (50 mL) under ice cooling. After 15 min, ammonium sulfamate (1.58 g, 13.8 mmol) was added and the resulting mixture was stirred for 15 min. After adjusting the pH to pH 5 using sodium acetate, 1,3-phenylenediamine (1 g, 9.2 mmol) was added; the mixture was further stirred for 2 h and then basified to pH 9 using 1M sodium hydroxide. Ethyl acetate was added and the organic layer washed twice with brine. The combined organic layers were dried over anhydrous MgSO₄ and evaporated to afford a red solid. A solution of copper sulfate (10 g) in aqueous ammonia (30 mL of 28% ammonia in 30 mL of water) was added to the previously obtained red solid in pyridine (40 mL). The solution was then refluxed for 16 h. After cooling, ethyl acetate was added, and the organic layer washed twice with brine. The combined organic layers were dried over anhydrous MgSO₄ and evaporated down to get a dark red solid, which was triturated with diethyl ether to afford 1.09 g (42%) of the title compound (LCMS RT=7.06 min, MH⁺ 282.1)

¹H NMR (DMSO): 8.02 (2H, d, J 9.3 Hz), 7.68 (1H, d, J 9.1 Hz), 6.96 (1H, dd, J 9.1 2.0 Hz), 6.86 (2H, d, J 9.3 Hz), 6.75 (1H, dd, J 1.9 0.6 Hz), 5.55 (2H, br), 3.46 (4H, q, J 7.1 Hz), 1.19 (6H, t, J 7.1 Hz)

All compounds below were prepared following the same general procedure and purified either by trituration with diethyl ether or by column chromatography on silica gel eluting with a gradient of ethyl acetate/hexanes.

### 6-Methyl-2-(4-morpholinophenyl)-2H-benzo[d][1,2,3]triazol-5-amine

LCMS RT=5.95 min, MH⁺ 311.9; ¹H NMR (DMSO): 8.03 (2H, d, J 9.2 Hz), 7.55 (1H, s), 7.11 (2H, d, J 9.3 Hz), 6.81 (1H, s), 5.32 (2H, s), 3.78-3.75 (4H, m), 3.19-3.16 (4H, m), 2.26 (3H, s)

### 2-(4-Chlorophenyl)-2H-benzo[d][1,2,3]triazol-5-amine

LCMS RT=6.72 min, MH⁺ 245.0; ¹H NMR (DMSO): 8.19 (2H, d, J 9.0 Hz), 7.69 (1H, d, J 9.4 Hz), 7.66 (2H, d, J 9.1 Hz), 6.99 (1H, dd, J 9.1 2.0 Hz), 6.68 (1H, d, J 1.9 Hz), 5.71 (2H, s)

### 2-(4-(Piperidin-1-yl)phenyl)-2H-benzo[d][1,2,3]triazol-5-amine

LCMS RT=7.21 min, MH⁺ 294.2; ¹H NMR (DMSO): 7.99 (2H, d, J 9.2 Hz), 7.65 (1H, d, J 9.2 Hz), 7.08 (2H, d, J 9.2 Hz), 6.92 (1H, dd, J 9.0 1.9 Hz), 6.70-6.69 (1H, m), 5.53 (2H, s), 3.28-3.23 (4H, m), 1.68-1.54 (6H, m)

### 2-(4-(Dimethylamino)phenyl)-2H-benzo[d][1,2,3]triazol-5-amine

LCMS RT=6.13 min, MH⁺ 254.1; ¹H NMR (DMSO): 7.99 (2H, d, J 9.2 Hz), 7.64 (1H, d, J 9.2 Hz), 6.91 (1H, dd, J 9.0 2.0 Hz), 6.86 (2H, d, J 9.2 Hz), 6.70 (1H, d, J 1.5 Hz), 5.50 (2H, s), 2.99 (6H, s)

### 2-(4-(4-Methylpiperazin-1-yl)phenyl)-2H-benzo[d][1,2,3]triazol-5-amine

LCMS RT=4.86 min, MH⁺ 309.1; ¹H NMR (DMSO): 8.01 (2H, d, J 9.2 Hz), 7.65 (1H, d, J 9.2 Hz), 7.10 (2H, d, J 9.2 Hz), 6.93 (1H, dd, J 9.0 1.9 Hz), 6.70-6.69 (1H, m), 5.54 (2H, s), 2.23 (4H, s)

### 2-(4-Chlorophenyl)-6-methyl-2H-benzo[d][1,2,3]triazol-5-amine

LCMS RT=7.13 min, MH⁺ 259.0; ¹H NMR (DMSO): 8.19 (2H, d, J 8.9 Hz), 7.65 (2H, d, J 8.9 Hz), 7.60-7.59 (1H, m), 6.80 (1H, s), 5.48 (2H, s), 2.27 (3H, s)

Method 2: Compounds II

### N-(2-(4-Chlorophenyl)-6-methyl-2H-benzo[d][1,2,3]triazol-5-yl)nicotinamide

To a solution of 2-(4-chlorophenyl)-6-methyl-2H-benzo[d][1,2,3]triazol-5-amine (50 mg, 0.19 mmol) and triethylamine (108 μL, 0.77 mmol) in dichloromethane (4 mL) was added 3-nicotinoyl chloride hydrochloride (38 mg, 0.21 mmol). The resulting mixture was stirred at room temperature overnight. Dichloromethane was added and the organic layer was washed twice with aqueous saturated Na₂CO₃. The combined organic layers were dried over anhydrous MgSO₄ and evaporated. The resulting solid was washed with diethyl ether to afford 7 mg (10%) of the title compound (LCMS RT=6.30 min, MH⁺ 364.2)

¹H NMR (DMSO): 10.26 (1H, s), 9.20 (1H, m), 8.82-8.79 (1H, m), 8.39-8.32 (3H, m), 8.13 (1H, s), 7.96 (1H, s), 7.74 (2H, d, J 8.9 Hz), 7.65-7.57 (1H, m)

All compounds below were prepared following the same general procedure.

### N-(2-(4-Chlorophenyl)-6-methyl-2H-benzo[d][1,2,3]triazol-5-yl)isonicotinamide

LCMS RT=6.37 min, MH⁺ 364.0; ¹H NMR (DMSO): 10.33 (1H, s), 8.83 (2H, d, J 6.0 Hz), 8.33 (2H, d, J 8.8 Hz), 8.12 (1H, s), 7.96-7.92 (3H, m), 7.73 (2H, d, J 8.9 Hz)

### N-(2-(4-Chlorophenyl)-6-methyl-2H-benzo[d][1,2,3]triazol-5-yl)benzamide

LCMS RT=7.63 min, MH⁺ 363.1; ¹H NMR (DMSO): 10.05 (1H, s), 8.33 (2H, d, J 9.1 Hz), 8.10 (1H, s), 8.04-7.94 (3H, m), 7.73 (2H, d, J 9.1 Hz), 7.64-7.55 (3H, m)

### N-(2-(4-Chlorophenyl)-6-methyl-2H-benzo[d][1,2,3]triazol-5-yl)-4-methoxybenzamide

LCMS RT=7.63 min, MH⁺ 392.7; ¹H NMR (DMSO): 9.88 (1H, s), 8.32 (2H, d, J 9.1 Hz), 8.08 (1H, s), 8.02 (2H, d, J 8.8 Hz), 7.93 (1H, s), 7.73 (2H, d, J 9.0 Hz), 7.09 (2H, d, J 8.8 Hz), 3.86 (3H, s)

### N-(2-(4-Chlorophenyl)-6-methyl-2H-benzo[d][1,2,3]triazol-5-yl)-2-methoxybenzamide

LCMS RT=9.42 min; ¹H NMR (DMSO): 10.23 (1H, s), 8.78 (1H, s), 8.32 (2H, d, J 9.0 Hz), 8.07-8.05 (1H, m), 7.96 (1H, s), 7.72 (2H, d, J 9.0 Hz), 7.66-7.59 (1H, m), 7.33-7.15 (2H, m), 4.08 (3H, s)

### N-(2-(4-Chlorophenyl)-6-methyl-2H-benzo[d][1,2,3]triazol-5-yl)thiophene-2-carboxamide

LCMS RT=7.44 min, MH⁺ 369.0; ¹H NMR (DMSO): 10.07 (1H, s), 8.32 (2H, d, J 9.1 Hz), 8.05-8.03 (2H, m), 7.95 (1H, s), 7.90 (1H, dd, J 5.0 1.0 Hz), 7.72 (2H, d, J 8.9 Hz), 7.28-7.25 (1H, m), 2.45 (3H, s)

### N-(2-(4-Chlorophenyl)-6-methyl-2H-benzo[d][1,2,3]triazol-5-yl)propionamide

LCMS RT=6.86 min, MH⁺ 315.2; ¹H NMR (DMSO): 9.48 (1H, s), 8.47 (2H, d, J 8.9 Hz), 8.32 (1H, s), 8.03 (1H, s), 7.88 (2H, d, J 8.9 Hz), 2.59 (3H, s), 1.30 (3H, t, J 7.1 Hz)

### N-(2-(4-Chlorophenyl)-6-methyl-2H-benzo[d][1,2,3]triazol-5-yl)butyramide

LCMS RT=7.32 min, MH⁺ 329.1; ¹H NMR (DMSO): 9.34 (1H, s), 8.29 (2H, d, J 8.9 Hz), 8.13 (1H, s), 7.86 (1H, s), 7.71 (2H, d, J 8.9 Hz), 2.42 (3H, s), 1.66-1.60 (2H, m), 0.97 (3H, t, J 7.1 Hz)

### N-(2-(4-Chlorophenyl)-6-methyl-2H-benzo[d][1,2,3]triazol-5-yl)pentanamide

LCMS RT=7.82 min, MH⁺ 343.2; ¹H NMR (DMSO): 9.34 (1H, s), 8.30 (2H, d, J 8.9 Hz), 8.13 (1H, s), 7.86 (1H, s), 7.71 (2H, d, J 8.9 Hz), 2.41 (3H, s), 1.66-1.58 (2H, m), 1.42-1.33 (2H, m), 0.94 (3H, t, J 7.1 Hz)

### N-(2-(4-Chlorophenyl)-6-methyl-2H-benzo[d][1,2,3]triazol-5-yl)isobutyramide

LCMS RT=7.23 min, MH⁺ 329.2; ¹H NMR (DMSO): 9.31 (1H, s), 8.30 (2H, d, J 8.9 Hz), 8.09 (1H, s), 7.87 (1H, s), 7.71 (2H, d, J 8.9 Hz), 2.77-2.73 (1H, m), 2.41 (3H, s), 1.17 (6H, d, J 6.8 Hz)

N-(2-(4-Chlorophenyl)-6-methyl-2H-benzo[d][1,2,3]triazol-5-yl)furan-2-carboxamide LCMS RT=7.44 min, MH⁺ 353.1; ¹H NMR (DMSO): 9.89 (1H, s), 8.32 (2H, d, J 9.1 Hz), 8.10 (1H, s), 7.98-7.93 (2H, m), 7.73 (2H, d, J 8.9 Hz), 7.36 (1H, dd, J 3.5 0.8 Hz), 6.74 (1H, dd, J 3.5 1.8 Hz), 2.44 (3H, s)

N-(2-(4-(Diethylamino)phenyl)-6-methyl-2H-benzo[d][1,2,3]triazol-5-yl)nicotinamide LCMS RT=6.94 min, MH⁺ 401.0; ¹H NMR (DMSO): 10.23 (1H, s), 9.20 (1H, m), 8.81-8.78 (1H, m), 8.39-8.33 (1H, m), 8.08 (2H, d, J 9.1 Hz), 8.01 (1H, s), 7.88-7.86 (1H, m), 7.63-7.56 (1H, m), 6.85 (2H, d, J 9.2 Hz), 3.43-3.40 (4H, m), 1.15 (6H, t, J 6.7 Hz)

### N-(2-(4-(Diethylamino)phenyl)-6-methyl-2H-benzo[d][1,2,3]triazol-5-yl)isonicotinamide

LCMS RT=6.99 min, MH⁺ 400.9; ¹H NMR (DMSO): 10.31 (1H, s), 8.82 (2H, d, J 6.0 Hz), 8.08 (2H, d, J 9.2 Hz), 8.00 (1H, s), 7.93 (2H, d, J 5.9 Hz), 7.89-7.87 (1H, m), 6.85 (2H, d, J 9.2 Hz), 3.42-3.38 (4H, m), 2.43 (3H, s), 1.15 (6H, t, J 7.2 Hz)

### N-(2-(4-(Diethylamino)phenyl)-6-methyl-2H-benzo[d][1,2,3]triazol-5-yl)propionamide

LCMS RT=7.51 min, MH⁺ 352.2; ¹H NMR (DMSO): 9.28 (1H, s), 8.06-8.02 (3H, m), 7.78 (1H, s), 6.83 (2H, d, J 9.2 Hz), 3.42-3.37 (6H, m), 2.38 (3H, s), 1.16-1.10 (9H, m)

N-(2-(4-(Diethylamino)phenyl)-6-methyl-2H-benzo[d][1,2,3]triazol-5-yl)butyramide LCMS RT=7.97 min, MH⁺ 366.1; ¹H NMR (DMSO): 9.32 (1H, s), 8.04 (2H, d, J 8.9 Hz), 8.00 (1H, s), 7.78 (1H, s), 6.84 (2H, d, J 8.9 Hz, 1.70-1.62 (2H, m), 1.17-1.07 (6H, m), 0.97 (3H, t, J 7.1 Hz)

N-(2-(4-(Diethylamino)phenyl)-6-methyl-2H-benzo[d][1,2,3]triazol-5-yl)pentanamide LCMS RT=8.53 min, MH⁺ 379.9; ¹H NMR (DMSO): 9.32 (1H, s), 8.04 (2H, d, J 8.9 Hz), 8.00 (1H, s), 7.78 (1H, s), 6.83 (2H, d, J 8.9 Hz), 3.50-3.35 (6H, m), 2.39 (3H, s), 1.65-1.61 (2H, m), 1.41-1.34 (2H, m), 1.17-1.07 (6H, m), 0.94 (3H, t, J 7.1 Hz)

### N-(2-(4-(Diethylamino)phenyl)-6-methyl-2H-benzo[d][1,2,3]triazol-5-yl)isobutyramide

LCMS RT=7.92 min, MH⁺ 366.1; ¹H NMR (DMSO): 9.29 (1H, s), 8.05 (2H, d, J 8.9 Hz), 7.96 (1H, s), 7.78 (1H, s), 6.84 (2H, d, J 8.9 Hz), 3.42-3.37 (4H, m), 2.41 (3H, s), 1.17-1.07 (12H, m)

### N-(2-(4-(Diethylamino)phenyl)-6-methyl-2H-benzo[d][1,2,3]triazol-5-yl)furan-2-carboxamide

LCMS RT=8.18 min, MH⁺ 389.9; ¹H NMR (DMSO): 9.87 (1H, s), 8.07 (2H, d, J 9.1 Hz), 7.98 (1H, s), 7.98-7.96 (1H, m), 7.85 (1H, m), 7.34 (1H, dd, J 3.4 0.7 Hz), 6.87 (2H, d, J 9.2 Hz), 6.73 (1H, dd, J 3.5 1.7 Hz), 3.42-3.37 (4H, m), 2.41 (3H, s), 1.17-1.07 (6H, m)

### N-(2-(4-(Diethylamino)phenyl)-2H-benzo[d][1,2,3]triazol-5-yl)nicotinamide

LCMS RT=7.19 min, MH⁺ 387.1; ¹H NMR (DMSO): 10.69 (1H, s), 9.16 (1H, s), 8.82-8.77 (1H, m), 8.54 (1H, s), 8.34 (1H, d, J 7.8 Hz), 8.08 (2H, d, J 9.3 Hz), 7.98 (1H, d, J 9.3 Hz), 7.71 (1H, dd, J 9.1 1.7 Hz), 7.58-7.53 (1H, m), 6.85 (2H, d, J 9.2 Hz), 3.44 (4H, q, J 6.8 Hz), 1.18 (6H, t, J 6.8 Hz)

### N-(2-(4-(Diethylamino)phenyl)-2H-benzo[d][1,2,3]triazol-5-yl)isonicotinamide

LCMS RT=7.23 min, MH⁺ 387.1; ¹H NMR (DMSO): 10.74 (1H, s), 8.82 (2H, d, J 5.6 Hz), 8.58-8.53 (1H, m), 8.08 (2H, d, J 9.4 Hz), 7.98 (1H, dd, J 9.1 0.6 Hz), 7.91 (2H, d, J 6.1 Hz), 7.71 (1H, dd, J 9.1 1.8 Hz), 6.86 (2H, d, J 9.1 Hz), 3.43 (4H, q, J 7.1 Hz), 1.15 (6H, t, J 7.1 Hz)

### N-(2-(4-(Diethylamino)phenyl)-2H-benzo[d][1,2,3]triazol-5-yl)acetamide

LCMS RT=6.89 min, MH⁺ 324.2; ¹H NMR (DMSO): 10.20 (1H, s), 8.38 (1H, d, J 1.8 Hz), 8.04 (2H, d, J 9.2 Hz), 7.89 (1H, dd, J 9.1 0.6 Hz), 7.42 (1H, dd, J 9.2 1.8 Hz), 6.84 (2H, d, J 9.2 Hz), 3.43 (4H, q, J 6.9 Hz), 2.11 (3H, s), 1.14 (6H, t, J 6.9 Hz)

### N-(2-(4-(Diethylamino)phenyl)-2H-benzo[d][1,2,3]triazol-5-yl)propionamide

LCMS RT=7.50 min, MH⁺ 338.2; ¹H NMR (DMSO): 10.12 (1H, s), 8.41 (1H, d, J 1.0 Hz), 8.04 (2H, d, J 9.2 Hz), 7.89 (1H, d, J 9.2 Hz), 7.44 (1H, dd, J 9.1 1.8 Hz), 6.84 (2H, d, J 9.4 Hz), 3.42 (4H, q, J 6.9 Hz), 2.39 (2H, q, J 7.4 Hz), 1.17-1.09 (9H, m)

### N-(2-(4-(Diethylamino)phenyl)-2H-benzo[d][1,2,3]triazol-5-yl)butyramide

LCMS RT=8.00 min, MH⁺ 352.1; ¹H NMR (DMSO): 10.13 (1H, s), 8.42-8.40 (1H, m), 8.04 (2H, d, J 9.2 Hz), 7.89 (1H, d, J 9.2 Hz), 7.44 (1H, dd, J 9.1 1.7 Hz), 6.84 (2H, d, J 9.4 Hz), 3.42 (4H, q, J 6.9 Hz), 2.36 (2H, q, J 7.4 Hz), 1.72-1.60 (2H, m), 1.14 (6H, t, J 7.0 Hz), 0.95 (3H, t, J 7.4 Hz)

### N-(2-(4-(Diethylamino)phenyl)-2H-benzo[d][1,2,3]triazol-5-yl)pentanamide

LCMS RT=8.60 min, MH⁺ 365.9; ¹H NMR (DMSO): 10.13 (1H, s), 8.42-8.40 (1H, m), 8.04 (2H, d, J 9.2 Hz), 7.89 (1H, d, J 9.2 Hz), 7.44 (1H, dd, J 9.1 1.7 Hz), 6.84 (2H, d, J 9.4 Hz), 3.42 (4H, q, J 6.9 Hz), 2.38 (2H, q, J 7.4 Hz), 1.67-1.57 (2H, m), 1.42-130 (2H, m), 1.14 (6H, t, J 7.0 Hz), 0.92 (3H, t, J 7.4 Hz)

### N-(2-(4-(Diethylamino)phenyl)-2H-benzo[d][1,2,3]triazol-5-yl)isobutyramide

LCMS RT=7.95 min, MH⁺ 352.2; ¹H NMR (DMSO): 10.09 (1H, s), 8.42-8.35 (1H, m), 8.04 (2H, d, J 9.2 Hz), 7.89 (1H, d, J 9.2 Hz), 7.46 (1H, dd, J 9.1 1.8 Hz), 6.84 (2H, d, J 9.4 Hz), 3.42 (4H, q, J 6.9 Hz), 2.70-2.61 (1H, m), 1.18-1.12 (12H, m)

N-(2-(4-(Diethylamino)phenyl)-2H-benzo[d][1,2,3]triazol-5-yl)furan-2-carboxamide LCMS RT=7.98 min, MH⁺ 376.3; ¹H NMR (DMSO): 10.43 (1H, s), 8.48-8.47 (1H, m), 8.07 (2H, d, J 9.2 Hz), 7.99-7.98 (1H, m), 7.94 (1H, d, J 9.2 Hz), 7.74 (1H, dd, J 9.3 1.9 Hz), 7.40 (1H, d, J 3.5 Hz), 6.85 (2H, d, J 9.3 Hz), 6.75-6.72 (1H, m), 3.43 (4H, q, J 7.1 Hz), 1.15 (6H, t, J 7.0 Hz)

### N-(2-(4-(Diethylamino)phenyl)-2H-benzo[d][1,2,3]triazol-5-yl)thiophene-2-carboxamide

LCMS RT=8.47 min, MH⁺ 391.9; ¹H NMR (DMSO): 10.45 (1H, s), 8.46-8.45 (1H, m), 8.09-8.06 (3H, m), 7.96 (1H, d, J 9.3 Hz), 7.91 (1H, dd, J 5.0 1.0 Hz), 7.70 (1H, dd, J 9.2 1.8 Hz), 7.28-7.25 (1H, m), 6.84 (2H, d, J 9.4 Hz), 3.43 (4H, q, J 7.0 Hz), 1.15 (6H, t, J 7.0 Hz)

### N-(2-(4-(Diethylamino)phenyl)-2H-benzo[d][1,2,3]triazol-5-yl)benzamide

LCMS RT=8.62 min, MH⁺ 385.9; ¹H NMR (DMSO): 10.50 (1H, s), 8.54-8.53 (1H, m), 8.08 (2H, d, J 9.2 Hz), 8.02-7.99 (2H, m), 7.96 (1H, dd, J 9.1 0.6 Hz), 7.74 (1H, dd, J 9.2 1.8 Hz), 7.67-7.54 (3H, m), 6.85 (2H, d, J 9.3 Hz), 3.44 (4H, q, J 7.0 Hz), 1.15 (6H, t, J 7.0 Hz)

N-(2-(4-(Diethylamino)phenyl)-2H-benzo[d][1,2,3]triazol-5-yl)-4-methoxybenzamide LCMS RT=8.58 min, MH⁺ 416.2; ¹H NMR (DMSO): 10.33 (1H, s), 8.52-8.51 (1H, m), 8.06 (2H, d, J 9.2 Hz), 8.01 (2H, d, J 8.8 Hz), 7.94 (1H, d, J 9.2 Hz), 7.73 (1H, dd, J 9.2 1.8 Hz), 7.09 (2H, d, J 8.8 Hz), 6.85 (2H, d, J 9.3 Hz), 3.86 (3H, s), 3.43 (4H, q, J 7.0 Hz), 1.15 (6H, t, J 7.0 Hz)

N-(2-(4-(Diethylamino)phenyl)-2H-benzo[d][1,2,3]triazol-5-yl)-2-methoxybenzamide LCMS RT=9.56 min, MH⁺ 415.9; ¹H NMR (DMSO): 10.40 (1H, s), 8.56-8.55 (1H, m), 8.07 (2H, d, J 9.2 Hz), 7.93 (1H, d, J 9.2 Hz), 7.67 (1H, dd, J 7.5 1.6 Hz), 7.61 (1H, dd, J 9.1 1.7 Hz), 7.56-7.51 (1H, m), 7.21 (1H, d, J 8.6 Hz), 7.10 (1H, t, J 7.5 Hz), 6.85 (2H, d, J 9.3 Hz), 3.93 (3H, s), 3.43 (4H, q, J 7.0 Hz), 1.15 (6H, t, J 7.0 Hz)

### 4-Chloro-N-(2-(4-(diethylamino)phenyl)-2H-benzo[d][1,2,3]triazol-5-yl)benzamide

LCMS RT=9.71 min, MH⁺ 420.0; ¹H NMR (DMSO): 10.56 (1H, s), 8.53-8.52 (1H, m), 8.08 (2H, d, J 9.2 Hz), 8.04 (2H, d, J 8.7 Hz), 7.96 (1H, d, J 9.1 Hz), 7.72 (1H, dd, J 9.2 1.8 Hz), 7.65 (2H, d, J 8.6 Hz), 6.85 (2H, d, J 9.2 Hz), 3.44 (4H, q, J 7.0 Hz), 1.15 (6H, t, J 7.0 Hz)

### N-(2-(4-(Diethylamino)phenyl)-2H-benzo[d][1,2,3]triazol-5-yl)-4-(dimethylamino)benzamide

LCMS RT=8.84 min, MH⁺ 428.9; ¹H NMR (DMSO): 10.02 (1H, s), 8.43-8.42 (1H, m), 7.99 (2H, d, J 9.2 Hz), 7.85-7.82 (3H, m), 7.66 (1H, dd, J 9.1 1.7 Hz), 6.77 (2H, d, J 9.4 Hz), 6.71 (2H, d, J 9.1 Hz), 3.35 (4H, q, J 7.0 Hz), 2.94 (6H, s), 1.07 (6H, t, J 7.0 Hz)

### N-(2-(4-Chlorophenyl)-2H-benzo[d][1,2,3]triazol-5-yl)propionamide

LCMS RT=7.16 min, MH⁺ 301.0; ¹H NMR (DMSO): 10.22 (1H, s), 8.50-8.48 (1H, m), 8.30 (2H, d, J 9.0 Hz), 7.97 (1H, d, J 9.3 Hz), 7.71 (2H, d, J 9.0 Hz), 7.50 (1H, dd, J 9.3 1.7 Hz), 1.13 (3H, t, J 7.1 Hz)

### N-(2-(4-Chlorophenyl)-2H-benzo[d][1,2,3]triazol-5-yl)butyramide

LCMS RT=7.64 min, MH⁺ 314.8; ¹H NMR (DMSO): 10.22 (1H, s), 8.49-8.48 (1H, m), 8.29 (2H, d, J 9.0 Hz), 7.97 (1H, d, J 9.3 Hz), 7.71 (2H, d, J 9.0 Hz), 7.51 (1H, dd, J 9.3 1.7 Hz), 2.38 (2H, t, J 7.0 Hz), 1.72-1.59 (2H, m), 0.94 (3H, t, J 7.4 Hz)

### N-(2-(4-Chlorophenyl)-2H-benzo[d][1,2,3]triazol-5-yl)isobutyramide

LCMS RT=7.59 min, MH⁺ 314.9; ¹H NMR (DMSO): 10.18 (1H, s), 8.50-8.49 (1H, m), 8.30 (2H, d, J 9.0 Hz), 7.97 (1H, d, J 9.3 Hz), 7.71 (2H, d, J 9.0 Hz), 7.53 (1H, dd, J 9.3 1.7 Hz), 2.67 (1H, m), 1.15 (6H, d, J 6.8 Hz)

### N-(2-(4-Chlorophenyl)-2H-benzo[d][1,2,3]triazol-5-yl)acetamide

LCMS RT=6.52 min, MH⁺ 287.0; ¹H NMR (DMSO): 10.30 (1H, s), 8.47-8.45 (1H, m), 8.29 (2H, d, J 9.0 Hz), 7.98 (1H, d, J 9.3 Hz), 7.71 (2H, d, J 9.0 Hz), 7.49 (1H, dd, J 9.3 1.7 Hz), 2.13 (3H, s)

### N-(2-(3,4-Dichlorophenyl)-2H-benzo[d][1,2,3]triazol-5-yl)isobutyramide

LCMS RT=8.29 min, MH⁺ 349.1; ¹H NMR (DMSO): 10.20 (1H, s), 8.50 (1H, dd, J 1.8 0.7 Hz), 8.48 (1H, d, J 2.5 Hz), 8.27 (1H, dd, J 8.8 2.5 Hz), 7.98 (1H, dd, J 9.2 0.7 Hz), 7.92 (1H, d, J 8.8 Hz), 7.55 (1H, dd, J 9.3 1.8 Hz), 2.71-2.62 (1H, m), 1.15 (6H, d, J 6.8 Hz)

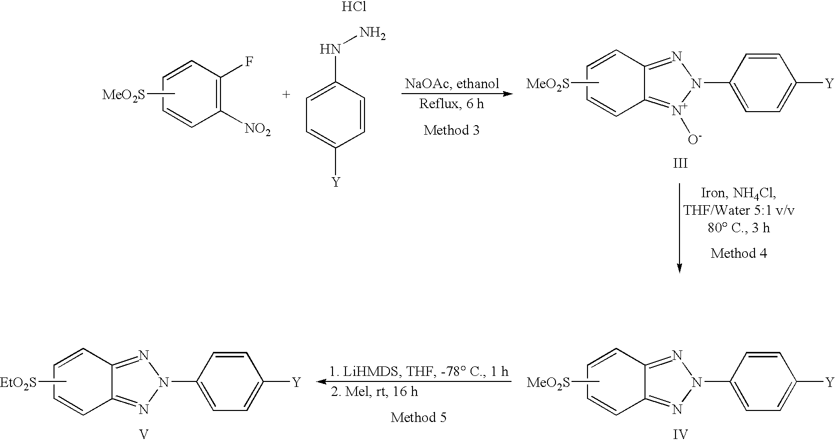

Method 3: Compounds III

### 2-(4-Chlorophenyl)-6-(methylsulfonyl)-2H-benzo[d][1,2,3]triazole 1-oxide

(4-Chlorophenyl)hydrazine hydrochloride (1.64 g, 9.17 mmol), 1-fluoro-4-(methylsulfonyl)-2-nitrobenzene (1.00 g, 4.56 mmol) and sodium acetate trihydrate (1.87 g, 13.7 mmol) were suspended in ethanol (15 mL) and heated to reflux for 6 h. The mixture was then cooled to room temperature and the product removed by filtration.

The residue was washed with methanol, water and then methanol again to afford 1.13 g (77%) of the title compound (LCMS RT=5.92 min, (MH⁺+MeCN) 364.9)

¹H NMR (DMSO): 8.39-8.38 (1H, m), 8.21-8.14 (3H, m), 7.98 (1H, dd, J 9.2 1.7 Hz), 7.80 (2H, d, J 9.0 Hz), 3.38 (3H, s)

### 6-(Methylsulfonyl)-2-(naphthalen-2-yl)-2H-benzo[d][1,2,3]triazole 1-oxide

LCMS RT=6.12 min; ¹H NMR (DMSO): 8.84 (1H, d, J 1.8 Hz), 8.42-8.41 (1H, m), 8.27-8.10 (5H, m), 8.01 (1H, dd, J 9.2 1.7 Hz), 7.76-7.68 (2H, m), 3.39 (3H, s)

Method 4: Compounds IV

### 2-(4-Chlorophenyl)-5-(methylsulfonyl)-2H-benzo[d][1,2,3]triazole

To a suspension of 2-(4-chlorophenyl)-6-(methylsulfonyl)-2H-benzo[d][1,2,3]triazole 1-oxide (157 mg, 0.49 mmol) and ammonium chloride (52 mg, 0.97 mmol) in tetrahydrofuran/water 5:1 v/v (6 mL) at 80° C. was added iron powder (136 mg, 2.43 mmol). The resulting mixture was stirred for 3 h at 80° C. After cooling, the solution was passed through a pad of Celite® and washed with tetrahydrofuran. The filtrate was then concentrated in vacuo, suspended in water and extracted three times with ethyl acetate. The combined organic layers were dried over anhydrous MgSO₄ and evaporated. The resulting solid was purified by column chromatography eluting with ethyl acetate/hexanes 25:75 v/v to afford 29.7 mg (20%) of the title compound (LCMS RT=6.59 min)

¹H NMR (CDCl₃): 8.60-8.58 (1H, m), 8.28 (2H, d, J 9.0 Hz), 8.04 (1H, dd, J 9.0 0.9 Hz), 7.82 (1H, dd, J 9.0 1.6 Hz), 7.49 (2H, d, J 9.0 Hz), 3.06 (3H, s)

The compound below was prepared following the same general procedure.

### 2-(3,4-Dichlorophenyl)-5-(methylsulfonyl)-2H-benzo[d][1,2,3]triazole

LCMS RT=7.35 min, MH⁺ 342.1; ¹H NMR (DMSO): 8.70-8.69 (1H, m), 8.57 (1H, d, J 2.5 Hz), 8.37-8.33 (2H, m), 8.04-7.97 (2H, m), 3.37 (3H, s)

### 5-(Methylsulfonyl)-2-(naphthalen-2-yl)-2H-benzo[d][1,2,3]triazole

LCMS RT=6.92 min; ¹H NMR (DMSO): 9.01 (1H, d, J 2.1 Hz), 8.73-8.72 (1H, m), 8.52 (1H, dd, J 8.9 2.2 Hz), 8.38 (1H, dd, J 9.0 0.8 Hz), 8.27 (2H, d, J 8.6 Hz), 8.13-8.08 (1H, m), 8.02 (1H, dd, J 9.0 1.7 Hz), 7.71-7.67 (2H, m), 3.38 (3H, s)

Method 5: Compounds V

### 2-(3,4-Dichlorophenyl)-5-(ethylsulfonyl)-2H-benzo[d][1,2,3]triazole

To a dry Schlenk flask under nitrogen was added 2-(3,4-dichlorophenyl)-5-(methylsulfonyl)-2H-benzo[d][1,2,3]triazole (93.5 mg, 0.27 mmol) and dry tetrahydrofuran (5 mL). The solution was then cooled down to −78° C., and lithium bis(trimethylsilyl)amide (0.30 mL, 0.30 mmol) was added. The reaction was left stirring at −78° C. for 1 h, and then methyl iodide (35 μL, 0.55 mmol) was added. The solution was allowed to warm up to room temperature for 16 h. Aqueous saturated ammonium chloride (10 mL) was added to the solution, the organic layer was separated and the aqueous layer was extracted three times with ethyl acetate. The combined organic layers were dried over anhydrous MgSO₄ and evaporated. The resulting solid was purified by column chromatography eluting with ethyl acetate/hexanes 20:80 v/v to afford 52 mg (54%) of the title compound (LCMS RT=7.65 min)

¹H NMR (DMSO): 8.68-8.67 (1H, m), 8.57 (1H, d, J 2.5 Hz), 8.38-8.33 (2H, m), 8.01-7.94 (2H, m), 3.46 (2H, q, J 7.5 Hz), 1.15 (3H, t, J 7.4 Hz)

The compound below was prepared following the same general procedure.

### 2-(4-Chlorophenyl)-5-(ethylsulfonyl)-2H-benzo[d][1,2,3]triazole

LCMS RT=6.89 min; ¹H NMR (DMSO): 8.67-8.66 (1H, m), 8.39 (2H, d, J 9.1 Hz), 8.34 (1H, dd, J 9.0 0.8 Hz), 7.95 (1H, dd, J 9.0 1.6 Hz), 7.79 (2H, d, J 9.0 Hz), 3.45 (2H, q, J 7.3 Hz), 1.15 (3H, t, J 7.4 Hz)

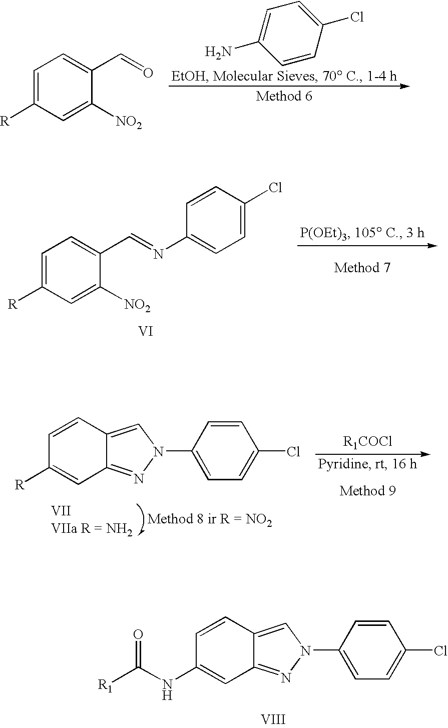

Method 6: Compounds VI

### (E)-4-Chloro-N-(4-(methylsulfonyl)-2-nitrobenzylidene)aniline

To 4-(methylsulfonyl)-2-nitrobenzaldehyde (250 mg, 1.09 mmol) in ethanol (5 mL) with molecular sieves at room temperature was added 4-chloroaniline (139 mg, 1.09 mmol). The resulting mixture was stirred at room temperature for 1 h, and then heated at 70° C. for 1 h. After cooling, the mixture was filtered off, and the filtrate concentrated in vacuo to afford the title compound, which was used crude in the next step.

Method 7: Compounds VII

### 2-(4-Chlorophenyl)-6-(methylsulfonyl)-2H-indazole

A suspension of (E)-4-Chloro-N-(4-(methylsulfonyl)-2-nitrobenzylidene)aniline (133 mg, 0.39 mmol) in triethyl phosphate (2 mL) was stirred at 105° C. for 3 h. After cooling, a solid was filtered off and washed with hexanes to afford 89 mg (74%) of the title compound (LCMS RT=6.17 min, MH⁺ 307.0)

¹H NMR (DMSO): 9.36 (1H, d, J 0.9 Hz), 8.34 (1H, br), 8.19 (2H, d, J 8.9 Hz), 8.08 (1H, dd, J 8.9 0.8 Hz), 7.73 (2H, d, J 8.9 Hz), 7.58 (1H, dd, J 8.8 1.4 Hz), 3.30 (3H, s)

The compound below was prepared following the same general procedure.

### 2-(4-Chlorophenyl)-6-nitro-2H-indazole

LCMS RT=7.27 min; ¹H NMR (DMSO): 9.40 (1H, s), 8.76-8.74 (1H, m), 8.20 (2H, d, J 9.0 Hz), 8.08 (1H, d, J 9.2 Hz), 7.89 (1H, dd, J 9.2 2.0 Hz), 7.74 (2H, d, J 8.9 Hz)

### 2-(4-Chlorophenyl)-2H-indazole

LCMS RT=7.05 min, MH⁺ 229.0; ¹H NMR (DMSO): 9.14 (1H, d, J 0.9 Hz), 8.14 (2H, d, J 9.0 Hz), 7.77 (1H, dt, J 8.4 1.1 Hz), 7.71 (1H, dd, J 8.8 0.9 Hz), 7.67 (2H, d, J=9.0 Hz), 7.33 (1H, ddd, J 8.9 6.6 1.1 Hz), 7.12 (1H, ddd, J 8.4 6.6 0.8 Hz)

Method 8: Compounds VIIa

### 2-(4-Chlorophenyl)-2H-indazol-6-amine

To 2-(4-chlorophenyl)-6-nitro-2H-indazole (103 mg, 0.37 mmol) in tetrahydrofuran:water 4:1 v/v (5 mL) at room temperature was added ammonium chloride (40 mg, 0.75 mmol). The mixture was heated at 80° C. and iron powder (105 mg, 1.87 mmol) was added. The resulting mixture was stirred at 80° C. for 3 h. After cooling, the solution was filtered through a pad of Celite® and washed with tetrahydrofuran. After evaporation of the solvent, the aqueous layer was extracted twice with ethyl acetate. The combined organic layers were washed with brine, dried over anhydrous MgSO₄ and evaporated to afford 84 mg (92%) of the title compound.

Method 9: Compounds VIII

### N-(2-(4-Chlorophenyl)-2H-indazol-6-yl)isobutyramide

To a solution of 2-(4-chlorophenyl)-2H-indazol-6-amine (84 mg, 0.34 mmol) in pyridine (5 mL) at room temperature was added isobutyryl chloride (43 μL, 0.41 mmol). The resulting mixture was stirred at room temperature for 16 h. Ethyl acetate was added and the organic layer was washed twice with saturated aqueous copper sulfate, followed by brine and water. The combined organic layers were dried over anhydrous MgSO₄ and evaporated. The resulting solid was purified by column chromatography eluting with ethyl acetate/hexanes 25:75 v/v to afford 11 mg (10%) of the title compound (LCMS RT=6.38 min, MH⁺ 314.2)

¹H NMR (DMSO): 10.14 (1H, s), 9.24 (1H, d, J 0.8 Hz), 8.42-8.40 (1H, m), 8.31 (2H, d, J 9.0 Hz), 7.89 (1H, dd, J 9.1 0.7 Hz), 7.85 (2H, d, J 8.9 Hz), 7.36 (1H, dd, J 9.0 1.6 Hz), 2.90-2.81 (1H, m), 1.34 (6H, d, J 6.8 Hz)

Method 10: Compounds IX

### 2-(4′-Chlorophenyl)-6-(isopropylsulfonyl)-2H-indazole

To a dry Schlenk flask under nitrogen was added 2-(4-chlorophenyl)-6-(methylsulfonyl)-2H-indazole (200 mg, 0.65 mmol) and dry tetrahydrofuran (9 mL). The solution was then cooled down to −78° C., and lithium bis(trimethylsilyl)amide (0.72 mL, 0.72 mmol) was added. The reaction was left stirring at −78° C. for 1 h, and then methyl iodide (81 μL, 1.31 mmol) was added. The solution was allowed to warm up to room temperature for 16 h. Aqueous saturated ammonium chloride (10 mL) was added to the solution, the organic layer was separated and the aqueous layer was extracted three times with ethyl acetate. The combined organic layers were dried over anhydrous MgSO₄ and evaporated. The resulting solid was purified by column chromatography eluting with ethyl acetate/hexanes 1:2 v/v to afford 20 mg (9%) of the title compound (LCMS RT=6.53 min, MH⁺ 335.2)

¹H NMR (DMSO): 9.37 (1H, d, J 0.9 Hz), 8.29-8.27 (1H, m), 8.18 (2H, d, J 9.0 Hz), 8.07 (1H, dd, J 8.8 0.8 Hz), 7.72 (2H, d, J 9.0 Hz), 7.49 (1H, dd, J 8.8 1.6 Hz), 3.58-3.48 (1H, m), 1.20 (6H, d, J 6.8 Hz)

The compounds listed in Table 2, can be prepared by analogues methods to those described above, or by literature methods known or adapted by the persons skilled in the art.

1. A method of treatment or prophylaxis of Duchenne muscular dystrophy, Becker muscular dystrophy or cachexia, comprising administering a compound of Formula (I) or (II) 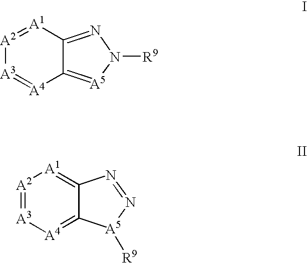
wherein
A¹, A², A³, A⁴ and A⁵ which may be the same or different, represent N or CR¹
R₉ represents -L-R³, in which L is a single bond or a linker group and R³ represents hydrogen or a substituent and
in addition,
when an adjacent pair of A¹-A⁴ each represent CR¹, then the adjacent carbon atoms, together with their substituents may form a ring B,
when A⁵ represents CR¹, then A⁵ and N—R⁹, together with their substituents may form a ring C,
or a pharmaceutically acceptable salt thereof.

2. The method according to claim 1, wherein R³ in the compound of formula I represents alkyl, alkoxy or aryl, each optionally substituted by one to three substitutents, R², which may be the same or different.

3. The method of claim 1, wherein
A⁵ represents N, wherein:
L is single bond and R³ represents:
thioalkyl optionally substituted by alkyl or optionally substituted aryl,
O-aryl or thioaryl, in which the aryl is optionally substituted,
optionally substituted aryl,
hydroxyl,
NR¹⁰R¹¹,
SO₂R¹²,
NR¹³SO₂R¹⁴,
C(═W)R¹⁶,
NR¹⁵C(═W)R¹⁷,
R¹⁰, R¹¹, R¹², R¹³, R¹⁴, R¹⁶ and R¹⁷, which may be the same or different, represent hydrogen, alkyl optionally substituted by optionally substituted aryl, optionally substituted aryl,
in addition,
R¹⁰ and R¹¹ together with the nitrogen to which they are attached may form a ring,
R¹² may have the same meaning as NR¹⁰R¹¹,
R¹⁶ and R¹⁷, which may be the same or different, may each represent
alkyl substituted by one or more of halogen, alkoxy optionally substituted aryl or optionally substituted aryl,
optionally substituted aryloxy,
aryl or NR¹⁰R¹¹,
and when R¹⁶ or R¹⁷ represents NR¹⁰R¹¹, one of R¹⁰ and R¹¹ may additionally represent CO alkyl optionally substituted or COaryl optionally substituted, and
in addition to the definitions shared with R¹⁷, R¹⁶ may represent hydroxyl;
or when the compound has formula II, then A⁵ represents CH, and
wherein L is single bond and R³ represents:
thioalkyl optionally substituted by alkyl or optionally substituted aryl,
thioaryl, in which the aryl is optionally substituted,
optionally substituted aryl,
hydroxyl,
NO₂,
CN,
NR¹⁰R¹¹,
halogen,
SO₂R¹²,
NR¹³SO₂R¹⁴,
C(═W)R¹⁶,
OC(═W)NR¹⁰R¹¹
NR¹⁵C(═W)R¹⁷,
R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,
in addition,
R¹⁰ and R¹¹ together with the nitrogen to which they are attached may form a ring,
R¹² may have the same meaning as NR¹⁰R¹¹,
R¹⁶ and R¹⁷, which may be the same or different, may each represent
alkyl substituted by one or more of halogen, alkoxy optionally substituted aryl or optionally substituted aryl,
optionally substituted aryloxy,
aryl or NR¹⁰R¹¹,
and when R¹⁶ or R¹⁷ represents NR¹⁰R¹¹, one of R¹⁰ and R¹¹ may additionally represent CO alkyl optionally substituted or COaryl optionally substituted, and
in addition to the definitions shared with R¹⁷, R¹⁶ may represent hydroxyl.

4. The method according to claim 2, in which R¹ and R², which may be the same or different, may represent:
alkyl optionally substituted by one or more halogen, alkoxy or optionally substituted aryl, thioaryl or aryloxy,
alkoxy optionally substituted by optionally by alkyl or optionally substituted aryl,
hydroxyl,
OC(═W)NR¹⁰R¹¹
aryl,
thioalkyl optionally substituted by alkyl or optionally substituted aryl,
thioaryl, in which the aryl is optionally substituted,
NO₂,
CN,
NR¹⁰R¹¹,
halogen,
SO₂R¹²,
NR¹³SO₂R¹⁴,
C(═W)R¹⁶,
NR¹⁵C(═W)R¹⁷,
P(═O)OR₄₀R₄₁,
R₁₀, R₁₁, R₁₂, R₁₃, R₁₄, R₁₅, R₁₆, R₁₇, R₄₀ and R₄₁, which may be the same or different, represent hydrogen, alkyl optionally substituted by optionally substituted aryl, optionally substituted aryl,
in addition,
NR¹⁰R¹¹ together with the nitrogen to which they are attached may form a ring,
R¹² may have the same meaning as NR¹⁰R¹¹,
when R¹⁷ represents NR¹⁰R¹¹, that NR¹⁰R¹¹ may represent hydrogen, COalkyl and CO optionally substituted aryl,
R¹⁶ may represent hydroxy, alkoxy, or NR¹⁰R¹¹,
and R¹⁷ may represent alkyl substituted by one or more of halogen, alkoxy, optionally substituted aryl or NR¹⁰R¹¹.

5. The method of claim 1, wherein
A⁵ represents N, wherein:
L represents a linker group which is:
O, S or NR¹⁸,
alkylene, alkenylene, alkynylene, each of which may be optionally interrupted by one or more of O, S, NR¹⁸, or one or more C—C single, double or triple bonds,
and R¹⁸ represents hydrogen, alkyl, COR¹⁶.
or when the compound has formula II, then A⁵ represents CH,
wherein:
L represents a linker group which is:
O, S, NR¹⁸,
alkylene, alkenylene, alkynylene, each of which may be optionally interrupted by one or more of O, S, NR¹⁸, or one or more C—C single, double or triple bonds,
a —N—N— single or double bond,
and R¹⁸ represents hydrogen, alkyl, COR¹⁶.

6. The method of, claim 1, in which when any of the substituents represents alkyl, alkyl is saturated and has from 1 to 10 carbon atoms.

7. The method of claim 1, in which aryl is an aromatic hydrocarbon or a 5 to 10 membered aromatic heterocyle containing 1 to 4 hetero atoms selected from an oxygen atom, a sulphur atom and a nitrogen atom as a ring constituent besides carbon.

8. The method of claim 1, in which aryl is phenyl or naphthalene.

9. The method of claim 1, in which aryl is furan, thiophene, pyrrole or pyridine.

10. The method according to claim 1, in which ring B or ring C is a saturated or unsaturated 3 to 10 membered carbocylic or heterocyclic ring.

11. The method according to claim 1, in which ring B is benzene ring.

12. The method according to claim 1, in which ring C is a 3-10 membered saturated or unsaturated carbocylic ring.

13. The method according to claim 1, in which at least one R₁ represents NR₁₅C(═W)R₁₇.

14. The method according to claim 1, in which at least one R₁ represents NR₁₅C(═O)R₁₇.

15. The method according to claim 1, in which at least one R₁ represents CONR₁₀R₁₁.

16. The method according to claim 1, in which at least one R₁ represents NHCOR₁₇, wherein R₁₇ is selected from:
alkyl C₁-C₆,
alkyl C₁-C₆ substituted by phenyl,
alkyl C₁-C₆ substituted by alkoxy C₁-C₆,
haloalkyl C₁-C₆,
perfluoroalkyl C₁-C₆,
phenyl optionally substituted by one or more of halogen, alkyl C₁-C₆, alkoxy C₁-C₆, amino, (alkyl C₁-C₆)amino, di(alkyl C₁-C₆)amino or phenyl,
CH:CH phenyl,
naphthyl, pyridinyl, thiophenyl and furanyl.

17. The method according to claim 1 in which one or both of R₁ and R₂ is other than —COOH.

18. The method according to claim 1, in which at least one of R₁ represents NR₁₅CONR₁₀R₁₁, wherein R₁₀ and R₁₁, which may be the same or different, are selected from optionally substituted aryl, alkyl and COaryl optionally substituted.

19. The method according to claim 1, in which at least one of R₁ represents NHCONHR₁₅ and R₁₅ is selected from phenyl, alkyl C₁ to C₆ and COphenyl optionally substituted by one or more halogen.

20. The method according to claim 1, in which at least one of R₁ represents alkyl C₁ to C₆, optionally substituted by phenyl or a 4 to 7-membered, preferably 5 or 6-membered saturated or unsaturated heterocycle preferably containing one to two heteroatoms selected from N, S and O.

21. The method according to claim 1 in which at least one of R₁ represents COR₁₆ and R₁₆ is alkoxy C₁-C₆, amino, (alkyl C₁-C₆)amino or di(alkyl C₁-C₆)amino.

22. The method according to claim 1, in which at least one of R₁ represents:
NO₂,
halogen,
amino or (alkyl C₁-C₆)amino or di(alkyl C₁-C₆)amino in which the alkyl C₁ to C₆ is optionally substituted by phenyl or a 5 or 6 membered saturated or unsaturated heterocycle,
NHSO₂alkyl C₁-C₆, NHSO₂phenyl,
SO₂alkyl C₁-C₆,
phenyl optionally substituted by C₁ to C₆ alkoxy C₁-C₆,
a 5-10 membered, saturated or unsaturated, mono- or bi-cyclic heterocycle containing from 1-3 heteroatoms selected from N, S and O.

23. The method according to claim 1, in which R₃ represent aryl and is optionally substituted by one to three substituents, R₂, which may be the same or different.

24. The method according to claim 22 in which R₃ is a 5-10 membered aromatic mono- or bi-cyclic system.

25. The method according to claim 23, in which the aromatic system is a hydrocarbon.

26. The method according to claim 24, in which the aromatic hydrocarbon is benzene or naphthalene.

27. The method according to claim 23, in which the aromatic system is a heterocyclic system containing up to three heteroatoms, which may be the same or different, selected from N, O and S.

28. The method according to claim 27, in which the heterocyclic system is thiophene, furan, pyridine or pyrrole.

29. The method according to claim 2, in which the substituent(s) R² is/are selected from is:
alkyl C₁-C₆, optionally substituted by thiophenyl or phenoxy, each optionally substituted by halogen,
alkoxy C₁-C₆,
phenyl,
thioalkyl C₁-C₆,
thiophenyl, optionally substituted by halogen,
NO₂,
CN
NR₁₀R₁₁, in which R₁₀ and R₁₁, which may be the same or different represent hydrogen, alkyl C₁-C₆, or together with the nitrogen to which they are attached form a 5 to 7 membered ring which may contain one or more additional heteroatoms selected from N, O and S,
halogen,
SO₂R₁₂, in which R₁₂ represents a 5 to 7 membered ring which may contain one or more additional heteroatoms selected from N, O and S,
NHCOR₁₇, in which R₁₇ represents
alkyl C₁-C₆, optionally substituted by:
phenyl or halogen, or
phenyl optionally substituted by alkoxy C₁-C₆, carboxy, or halogen, or
a 5 or 6 membered saturated or unsaturated heterocycle,
phenyl or a 5 or 6 membered saturated or unsaturated heterocycle optionally substituted by halogen, alkoxy C₁ to C₆, carboxy or a group SO₂NR₁₀R₁₁.

30. The method according to claim 29 in which NR₁₀ R₁₁ represents N-pyrrole, N-piperidine, N′(C₁-C₆) alkyl N piperazine or N-morpholine.

31. The method of claim 1 in which, in formula II, A⁵ represents CH, wherein L represents:
—NH.NH—,
—CH═CH—,
—C≡C— or
—NCOR₁₆ in which R₁₆ represents phenyl or a 5 or 6 membered saturated or unsaturated heterocycle optionally substituted by halogen, alkoxy C1 to C6, carboxy.

32. The method according to claim 1 in which two of A₁-A₄ represent nitrogen.

33. The method according to claim 1 in which one of A₁-A₄ represents nitrogen.

34. The method according to claim 1 in which all of A₁-A₄ represents CR₁.

35. The method according to claim 1, wherein the compound is listed in table 1.
